(19) United States
(12) Patent Application Publication
(10) Pub. No.: US 2015/0173333 A1
(43) Pub. Date: Jun. 25, 2015

(54) RAPID SPECIFIC PATHOGEN FREE ANIMAL

(71) Applicant: BIOVALENCE SDN. BHD., Petaling Jaya (MY)

(72) Inventors: Eng Huan Ung, Sabah (MY); Ag., Muhammad Sagaf Abu Bakar, Sabah (MY)

(73) Assignee: BIOVALENCE SDN. BHD., Petaling Jaya (MY)

(21) Appl. No.: 14/408,799

(22) PCT Filed: Jun. 25, 2013
(86) PCT No.: PCT/MY2013/000116

§ 371 Date: Dec. 17, 2014

(30) Foreign Application Priority Data
Jun. 26, 2012 (MY) PI 2012002924

(51) Int. Cl. A01K 67/033; C12N 15/62; C12N 15/85

(52) U.S. Cl. CPC A01K 67/0338; C12N 15/8509; C12N 15/62

(57) ABSTRACT

A method of producing at least one specific pathogen free (SPF) non-human animal and/or a method of producing at least one specific pathogen resistant (SPR) non-human animal, the method comprising administration of a fusion protein to the surviving animal wherein the fusion protein comprises at least one polypeptide B which is a Type 1 Ribosome Inactivating Protein (RIP) or fragment thereof; and (i) at least one polypeptide A which is an Antimicrobial peptide; and/or (ii) at least one polypeptide C which is a Cationic Antimicrobial Peptide (CAP) or fragment thereof.

### FIELD OF THE INVENTION

The present invention relates to methods of producing specific pathogen free and/or specific pathogen resistant animals.

### BACKGROUND TO THE INVENTION

Specific pathogen free (SPF) non-human animals are essential for research purposes and to maintain standardised health and farming. Technological advances in the past decade have led to more sensitive research and commercialization outcomes that are recognizably affected by the presence of unwanted microorganisms, especially viruses. Accordingly, there is a need to produce/farm animals which are free from these unwanted microorganisms (i.e. SPF animals).

Diseases are the bane of farmers everywhere, costing farmers large amounts of money annually. When one animal is affected by a disease, most animals in the vicinity or within the same farm will also be infected causing an epizootic that may be both dangerous to the health of the human being and/or costing the human being lots of money. Many problems related to diseases are preventable by exercising common sense and science-based animal-rearing strategies. One of these methods is by using SPF non-human animals. For example, Early. Mortality Syndrome (EMS) also known as Acute Hepatopancreatic Necrosis Syndrome (AHPNS) in shrimp typically manifests in the first 10-40 days after stocking in ponds. It began in China in 2009, spread to Vietnam in 2010, to Malaysia in 2011 and then to Thailand in 2012 with global losses exceeding USD1 billion annually. Before the recent EMS epizootic, the only other shrimp virus capable of causing losses exceeding USD1 billion annually was the White Spot Syndrome Virus (WSSV). The pathogen responsible for EMS has recently been identified as a strain of Vibrio parahaemolyticus bacteria that has been transferred a toxic gene via a specific bacteriophage.

A reason why SPF animals have become extremely important for example in aquaculture is because it is now commonplace to farm alien species that are not endemic to a particular nation and therefore, to prevent introduction of new microorganisms to a place, use of SPF animals are required. One example is the use of the fast growing shrimp species Penaeus vannamei which although originally of Latin American origin, is now farmed in almost every nation where shrimp farming is a major aquaculture activity, replacing the slower growing Tiger Shrimp Penaeus monodon. For P. vannamei, under commercial conditions in Asian earthen ponds, typical growth rates of 1.0-1.5 g/wk (with 80-90 percent survival) are common in the high-density pond system (60-150/m²) currently in use in Thailand and Indonesia. In contrast, the growth (and survival) rate of P. monodon has been declining in recent years from 1.2 to 1 g/wk (and 45 percent to 55 percent survival) over the last few years in Thailand. In order to increase the survival rate of at least the P. monodon, use of SPF versions will help.

Standard practices for the production of SPF animals for example in aquaculture use has been described as long back as 1994 and little has changed since in terms of the actual process. The main objective has been to provide disease-free fry, fingerlings and post larvae to aquaculture farms to reduce the risk of disease introduction causing widespread epizootics.

Genuine SPF shrimp, by present conventions, are those which are produced from bio secure facilities, have been repeatedly examined, tested and found free of specified pathogens using intensive surveillance protocols and molecular methods, and originate from brood stock developed with strict founder population development protocols. These founder populations are generated by extensive quarantine procedures that result in SPF F1 generations derived from wild parents. The history of domestication programmes in various countries, in that such stocks may have been deliberately in-bred and consists entirely of siblings. This means that future generations of animals based only on such lines will probably lead to inbreeding within a few generations. Such inbreeding has been noted in stocks of P. stylirostris bred in Tahiti for 22 generations. It has also been noted in captive stocks of P. vannamei, which were characterized by a diminished ability to tolerate Taura Syndrome virus (TSV) challenges compared to a more diverse, heterozygous wild control population.

Accordingly, although SPF animals have their advantages, producing them is a time-consuming process that may result in other problems such as inbreeding. Further the potential drawback of SPF animals is that they are only SPF for the specific diseases for which they have been checked. However, there is yet to be any SPF source of EMS-free brood stock shrimp or post larvae available globally.

There is thus a need in the art for not only a quicker way of obtaining a SPF animal but also a simpler method of producing Specific Pathogen Resistant (SPR) animals.

SPR describes a genetic trait of a shrimp that confers some resistance against one specific pathogen. SPR shrimp usually result from a specific breeding programme designed to increase resistance to a particular virus. SPF and SPR are independent characteristics. Not all SPR shrimp are SPF and vice versa. A selective breeding programme for P. vannamei was initiated in 1995 in the Oceanic Institute in Hawaii. Original work was based on a selection index weighted equally for growth and TSV resistance (the major disease problem in the Americas at that time). Confirmation that growth and survival (to TSV challenge) responded well to selection was obtained, but there appeared to be a negative genetic correlation between these traits. Further investigation revealed that the shrimp selected only for growth were 21 percent larger than unselected shrimp (24 vs. 20 g) after one generation, with a realized heritability (h2) of 1. Females were 12.7 percent larger than males at about 22 g, but it was not possible to select for a higher percentage of females. Meanwhile, shrimp selected on an index weighted 70 percent for TSV resistance and 30 percent for growth showed an 18 percent increase in survival to a TSV challenge (46 vs. 39 percent) after one generation, with a realized heritability (h2) of 0.28. However, selected shrimp were 5 percent smaller than control shrimp, revealing a negative genetic correlation between mean family growth and mean family survival to a TSV challenge. This negative correlation between growth and disease resistance must therefore be taken into account when developing breeding plans for these shrimp. Taura Syndrome Virus or TSV can cause significant losses in farms stocked with P. vannamei and can be transmitted easily through insect or avian vectors between ponds. Because of this, the use of TSV-resistant (TSV-SPR) strains combined with biosecurity measures to reduce infections with other viruses such as WSSV, IHHNV and YHV could greatly assist the development of the new culture industry for P. vannamei in Asia. Such a protocol was adopted by the United States of America industry that, as a result, has seen a 50 percent growth rate per year over the last few years.

In view of the above, SPF and SPR are both essential and improved methods of producing and breeding them is needed.

### SUMMARY OF THE INVENTION

The present invention is defined in the appended independent claims. Some optional features of the present invention are defined in the appended dependent claims.

According to one aspect of the present invention, there is provided a method of producing at least one specific pathogen free (SPF) non-human animal, the method comprising:
(a) selecting a surviving animal in an environment comprising at least one pathogen that is capable of infecting and/or killing the animal;
(b) administration of a fusion protein to the surviving animal wherein the fusion protein comprises at least one polypeptide B which is a Type 1 Ribosome Inactivating Protein (RIP) or fragment thereof; and
   (i) at least one polypeptide A which is an Antimicrobial peptide; and/or
   (ii) at least one polypeptide C which is a Cationic Antimicrobial Peptide (CAP) or fragment thereof; and
(c) resulting surviving animal is the SPF non-human animal.
   Step (c) may be confirmed using conventionally accepted molecular methods that show the absence of the pathogen in question.

In another aspect of the present invention, there is provided a method of producing at least one specific pathogen resistant (SPR) non-human animal, the method comprising:
(a) producing a specific pathogen free animal according to any method of the present invention; and
(b) selective breeding of a male and female SPF non-human animal to produce a SPR non-human animal offspring.
   Step (b) may occur at the first, second, third, forth, fifth or tenth generation in the procedure of selective breeding. In particular, step (b) may eventually occur.

According to a further aspect of the present invention, there is provided a specific pathogen free or resistant non-human animal produced by any method of the present invention.

As will be apparent from the following description, preferred embodiments of the present invention allow for a fusion protein with an optimal effectiveness with a broad spectrum therapy and/or allowing oral delivery of the protein as some of the several applications.

### BRIEF DESCRIPTION OF THE FIGURES

Preferred embodiments of the fusion protein will now be described by way of example with reference to the accompanying figures in which:

FIG. 1 is a translation map of RetroMAD1 (SEQ ID NO:1 and SEQ ID NO:2).

FIG. 2 has two photos of gels showing A) Time course expression and B) Solubility of RetroMAD1 expression in E. Coli BL21(DE3) cells. Cells harbouring pRMD were harvested before induction (Oh), and after induction for 1 h, 2 h and 3 h represents the pellet phase, the hours with asterisk (*) represents the supernatant phase. Proteins were analysed on a 15% SDS-PAGE. M: PageRuler™ Protein Ladder Fermentas, U: uninduced, IND: induced and IB: purified inclusion bodies. Arrow indicates E. coli produced RetroMAD1 (41.2 kDa).

FIG. 3 is a photo of an agarose gel showing the PCR products in particular, the expected band of 441 by confirming the absence of the virus in the RetroMAD1 treated prawns.

FIG. 4 shows the experimental set-up of Example 3 to test the effects of RetroMAD1 on WSSV.

FIG. 5 are graphs showing the results that RetroMAD1 treated prawns survived for a longer period of time compared to the control (i.e. WSSV infected prawns).

FIG. 6 are gel images of showing the stability of fusion proteins, RetroMAD1, RetroGAD1, Amatilin and Tamapal1: A1 and A2 are RetroMAD1 subjected to temperature fluctuations; B1 and B2 are RetroGAD1 subjected to temperatures; C1 and C2 are Amatilin subjected to temperature fluctuations; D1 and D2 are Tamapal1 subjected to temperature fluctuations. Protein Ladder is the marker for protein size; Control is untreated drug; T1-4 are the different temperature fluctuations (as shown in Table 6) BME is 2×β-mercaptoethanol, the samples are loaded with (+) or without (−) BME.

FIG. 7 is a graph showing the percentage of viral reduction caused by Amatilin, RetroGAD1 and Tamapal1 exposed to various temperature fluctuations in simultaneous treatment determined by PCR.

FIG. 8A-D are graphs showing concentration of RetroMAD1 (μg/ml) (A), RetroGAD1 (μg/ml) (B), Amatilin (μg/ml) (C), Tamapal1 (μg/ml) (D) leached out against Time (minutes)

FIG. 9 is a graph showing concentration of RetroMAD1 in hepatopancreas, tail muscle, faeces and control against time in a short-term pharmacokinetics study

FIG. 10 is a graph showing concentration of RetroMAD1 in hepatopancreas, tail muscle, faeces and control against time in a long-term pharmacokinetics study

FIGS. 11A and B is an image of plates showing the anti-bacterial activity of amatilin against V. cholera (A) and V. parahemolyticus (B).(Plate: 1. 322.5 μg/ml, 2. 161.25 μg/ml, 3. 80.63 μg/ml, 4. 40.31 μg/ml, 5. 20.16 μpg/ml, 6. 10.08 μg/ml, 7. 5.04 μg/ml, 8. 2.52 μg/ml, 9. Untreated)

FIG. 12 is a graph showing the percentage of viral reduction caused by Amatilin, RetroGAD1, RetroMAD1 and Tamapal1 in simultaneous treatment at 72 h determined by PCR.

FIG. 13 A-C is a graph showing the percentage of viral reduction caused by drugs (A: Amatilin; B: RetroGAD1, C: Tamapal1) incubated at different temperatures for 1, 7 and 30 days in simultaneous treatment determined by PCR. (* Thermostability was not tested for 50° C. for 30 days incubation)

FIG. 14 is a schematic diagram showing the Supercritical Fluid Drying (SCFD) Process

FIG. 15 is a Scanning Electron Microscope (SEM) image of RetroMAD1 crystals

FIG. 16 is a graph showing the percentage of viral reduction caused by RetroMAD1 micronized powder in simultaneous treatment determined by PCR.

### DETAILED DESCRIPTION OF THE INVENTION

For convenience, certain terms employed in the specification, examples and appended claims are collected here.

The term “adjuvant”, as used in the context of the invention refers to an immunological adjuvant. By this, an adjuvant is meant to be a compound that is able to enhance or facilitate the immune system's response to the ingredient in question, thereby inducing an immune response or series of immune responses in the subject. The adjuvant can facilitate the effect of the therapeutic composition by forming depots (prolonging the half-life of the ingredient), provide additional T-cell help and stimulate cytokine production. Facilitation of antigen survival and unspecific stimulation by adjuvants may, in some cases, be required if the antigenic molecule are only weakly antigenic or only exerts weak to moderate interactions with compounds, molecules, or cells of the immune system.

The term “analogue” as used in the context of the invention refers to a peptide that may be modified by varying the amino acid sequence to comprise one or more naturally-occurring and/or non-naturally-occurring amino acids, provided that the peptide analogue is capable of reducing or preventing growth of a tumour or cancer. For example, the term “analogue” encompasses an inhibitory peptide comprising one or more conservative amino acid changes. The term “analogue” also encompasses a peptide comprising, for example, one or more D-amino acids. Such an analogue has the characteristic of, for example, protease resistance. Analogues also include peptidomimetics, e.g., in which one or more peptide bonds have been modified. Preferred analogues include an analogues of a peptide as described according to any embodiment here comprising one or more non-naturally-occurring amino acid analogues.

The term “comprising” as used in the context of the invention refers to where the various components, ingredients, or steps, can be conjointly employed in practicing the present invention. Accordingly, the term “comprising” encompasses the more restrictive terms “consisting essentially of” and “consisting of.” With the term “consisting essentially of” it is understood that the epitope/antigen of the present invention “substantially” comprises the indicated sequence as “essential” element. Additional sequences may be included at the 5′ end and/or at the 3′ end. Accordingly, a polypeptide “consisting essentially of” sequence X will be novel in view of a known polypeptide accidentally comprising the sequence X. With the term “consisting of” it is understood that the polypeptide, polynucleotide and/or antigen according to the invention corresponds to at least one of the indicated sequence (for example a specific sequence indicated with a SEQ ID Number or a homologous sequence or fragment thereof).

The term “derivative” as used in the context of the invention includes e.g., a fragment or processed form of the stated peptide, a variant or mutant comprising one or more amino acid substitutions, deletions of additions relative to the stated peptide, a fusion protein comprising the stated peptide or a peptide comprising one or more additional non-peptide components relative to the stated peptide e.g., a chemical component, e.g., polyethylene glycol (PEG). The term “derivative” also encompasses polypeptides comprising the fusion protein according to the invention. For example, the polypeptide comprises a label, such as, for example, an epitope, e.g., a FLAG epitope or a V5 epitope or an HA epitope. For example, the epitope is a FLAG epitope. Such a tag is useful for, for example, purifying the polypeptide. A preferred derivative of an antitumour or anticancer fusion protein of the invention has enhanced stability. For example, a cleavage site of a protease active in a subject to which a fusion protein is to be administered is mutated and/or deleted to produce a stable derivative of an antitumour or anticancer fusion protein of the invention. The term “derivative” also encompasses a derivatized peptide, such as, for example, a peptide modified to contain one or more-chemical moieties other than an amino acid. The chemical moiety may be linked covalently to the peptide e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound.

Accordingly, acceptable amino acid substitutions are generally therefore based on the relative similarity of the amino acid side-chain substituents, for example, their hydrophobicity, hydrophilicity, charge, size, and the like. Exemplary substitutions which take several of the foregoing characteristics into consideration are well known to those of skill in the art and include: arginine and lysine; glutamate and aspartate; serine and threonine; glutamine and asparagine; and valine, leucine and isoleucine. The isolated peptides of the present invention can be prepared in a number of suitable ways known in the art including typical chemical synthesis processes to prepare a sequence of polypeptides.

The term “fragment” as used in the context of the invention refers to an incomplete or isolated portion of the full sequence of the fusion protein according to any aspect of the present invention which comprises the active site(s) that confers the sequence with the characteristics and function of the protein. In particular, it may be shorter by at least one amino acid. For example a fragment of the fusion protein according to the present invention comprises the active site(s) that enable the protein to recognise an aberrant cell such as a tumour cell or cancer cell. The fragment may at least be 10 amino acids in length. For example, a non-limiting fragment of RIP may at least comprise the core or the bioactive site of the RIP which may be approximately 5 kDa in size.

The term “fusion protein(s)” as used in the context of the invention refers to proteins created through the joining of two or more genes, which originally coded for separate proteins. Translation of this fusion gene results in a single polypeptide with functional properties derived from each of the original proteins. Recombinant fusion proteins are created artificially by recombinant DNA technology for use in biological research or therapeutics. For example, the fusion protein according to any aspect of the present invention may comprise a polypeptide B; and a polypeptide C which is a CAP. The fusion protein may have antiviral properties. The fusion protein according to any aspect of the present invention may further comprise a polypeptide A. Each individual part and/or the whole the fusion protein may have antiviral properties. For example, polypeptide A, B, and/or C may have anticancer properties. As a whole A-B-C may have antiviral properties. The structure of the fusion protein may be A-B-C, A-C-B, C-A-B, C-B-A, B-A-C, B-C-A, A-B-C-C, A-B, B-C, B-C-C, C-C-B-C-C, or C-B-C. In particular, the fusion protein may comprise dimers and/or tandem repeats. More in particular, the structure of the fusion protein according to any aspect of the present invention may be repeats of the structure mentioned above. For example, the structure may be A-A-B-C-C, C-C-B-C-C, A-A-B-A-A and the like. The polypeptide A, B or C in each fusion protein may be the same protein or may be a different protein when repeated. Polypeptide A may be theta defensin, an analogue, or a fragment thereof. A fusion protein according to the present invention may comprise the sequence of SEQ ID NO:1, a variant, derivative or fragment thereof. The term “RetroMAD1” is used in the present invention to refer to a fusion protein with the structure A-B-C and with amino acid sequence SEQ ID NO:1. In particular, in RetroMAD1 polypeptide A may be Retrocyclin 101, polypeptide B may be MAP30 and polypeptide C may be Dermaseptin 1. These peptides may be directly fused to one another or connected to one another by a linker peptide.

The term “linker peptide”, as used in the context of the invention is used interchangeably with the term “linker” herein. A linker peptide is a peptide that covalently or non-covalently connects two or more molecules or peptides, thereby creating a larger complex consisting of all molecules or peptides including the linker peptide. A non-limiting example of a linker peptide may be SEQ ID NO:3 and/or SEQ ID NO:27.

The term “pathogen” as used in the context of the invention may refer to any disease-producing agent, especially a virus, bacterium, or other microorganism. A virus may be selected from the group consisting of cytomegalovirus (CMV), Epstein-Barr virus (EBV), varicella zoster virus (VZV), HSV-1, HSV-2, HSV-6, BK-virus, influenza viruses, respiratory syncytial virus (RSV); human immunodeficiency virus (HIV), hepatitis A, B or C (HBV), polio viruses, enteroviruses, human coxsackie viruses, rhinoviruses, echoviruses, equine encephalitis viruses, rubella viruses, dengue viruses, encephalitis viruses, yellow fever, coronaviruses, vesicular stomatitis viruses, rabies viruses, ebola viruses, parainfluenza viruses, mumps virus, measles virus, Hanta viruses, bunga viruses, phleboviruses and Nairo viruses, hemorrhagic fever viruses, reoviruses, orbiviurses and rotaviruses, parvoviruses, papilloma viruses, polyoma viruses, adenoviruses, herpes simplex virus (HSV) 1 and HSV-2, varicella zoster virus, variola viruses, vaccinia viruses, pox viruses, African swine fever virus, Iridovirus, Infectious Salmonid Anaemia (ISA), White Spot Syndrome Virus (WSSV), Hepatopancreactic parvo-like virus (HPV), Monodon Baculo virus (MBV), Infectious Hypodermal and Hematopoietic Necrosis Virus (IHHNV), Yellow Head Virus (YHV), Taura syndrome virus (TSV), Gill-associated virus (GAV), Laem-Singh Virus (LSNV), Infectious Myonecrosis Virus (IMNV), Mourilyan virus (MoV), Koi herpesvirus 1 (KHV 1), KHV2, KHV3, viral nervous necrosis (VNN), infectious pancreatic necrosis virus (IPNV), channel catfish virus (CCV), fish lymphocystis disease virus (FLDV), infectious hematopoietic necrosis virus (IHNV) and viral hemorrhagic septicemia virus (VHSV), AVG, AMAV, swine hepatitis E virus, Circoviruses, Herpesviruses, Porcine cytomegalovirus, pseudorabies virus, Feline Panleukopenia virus (FPV), Feline herpesvirus, Feline calicivirus, Feline Leukemia Virus (FeLV), Feline Immunodeficiency Virus (FIV), Rabies virus, canine parvovirus, canine coronavirus, canine distemper virus, canine influenza, canine hepatitis virus, canine herpesvirus, a virus that causes pseudorabies, canine minute virus and the like.

In particular, the viruses may only be viruses that are capable of infecting a non-human animal. The virus may be selected from the group consisting of Avian influenza viruses, Lymphoid Leukosis, Visceral Leukosis (Marek's Disease), Quail Bronchitis viruses, Newcastle disease viruses, infectious bronchitis viruses, infectious Bursal disease viruses, rhinoviruses, echoviruses, equine encephalitis viruses, coronaviruses, vesicular stomatitis viruses, rabies viruses, ebola viruses, parainfluenza viruses, Hanta viruses, bunga viruses, phleboviruses and Nairo viruses, hemorrhagic fever viruses, reoviruses, orbiviurses and rotaviruses, parvoviruses, papilloma viruses, polyoma viruses, adenoviruses, Aquabirnaviruses, Betanoda viruses, Salmonid alphaviruses, Epizotic Hematopoietic necrosis viruses, Infectious salmon anemia viruses (ISAV), Nervous necrosis viruses, Abalone Viral ganglioneuritis, Abalone Herpes-like viruses, variola viruses, vaccinia viruses, pox viruses, African swine fever virus, Iridovirus, Infectious Salmonid Anaemia (ISA), White Spot Syndrome Virus (WSSV), Hepatopancreactic parvo-like virus (HPV), Monodon Baculo virus (MBV), Infectious Hypodermal and Hematopoietic Necrosis Virus (IHHNV), Yellow Head Virus (YHV), Taura syndrome virus (TSV), Gill-associated virus (GAV), Laem-Singh Virus (LSNV), Infectious Myonecrosis Virus (IMNV), Mourilyan virus (MoV), Koi herpesvirus 1 (KHV 1), KHV2, KHV3, viral nervous necrosis (VNN), infectious pancreatic necrosis virus (IPNV), channel catfish virus (CCV), fish lymphocystis disease virus (FLDV), infectious hematopoietic necrosis virus (IHNV) and viral hemorrhagic septicemia virus (VHSV), AMAV, swine hepatitis E virus, Circoviruses, Herpesviruses, Porcine cytomegalovirus, pseudorabies virus, Feline Panleukopenia virus (FPV), Feline herpesvirus, Feline calicivirus, Feline Leukemia Virus (FeLV), Feline Immunodeficiency Virus (FIV), Rabies virus, canine parvovirus, canine coronavirus, canine distemper virus, canine influenza, canine hepatitis virus, canine herpesvirus, a virus that causes pseudorabies, canine minute virus and the like.

A virus may include a bacteriophage, also known as a phage that includes a group of viruses that infect specific bacteria, usually causing their disintegration or dissolution. A bacteriophage may be selected from a group consisting of Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttavirus, Inoviridae, Leviviridae, Microviridae, Plasmaviridae, Tectiviridae and the like. In particular, the phage may be Lambda phage (γ phage)-lysogen (λ phage), T2 phage, T4 phage, T7 phage, T12 phage, R17 phage, M13 phage, MS2 phage, G4 phage, P1 phage, Enterobacteria phage P2, P4 phage, Phi X 174 phage, N4 phage, Pseudomonas phage φ6, φ29 phage, 186 phage and the like.

A bacteria may include Aeromonas hydrophila, Aeromonas salmonicida, Aeromonas sobrio, Enterobacter aerogenes, Enterococcus faecalis, Escherichia coli, Flavobacterium meningosepticum, Helicobacter pylori, Klebsiella pneumonia, Listeria monocytogenes, Listonella anguillarum, Methicillin-resistant Staphylococcus aureus, Micrococcus luteus, Morganella morganii, Pasturella multocida, Pseudomonas aeruginosa, Salmonella typhimurium, Staphylococcus aureus, Staphylococcus epidermidis, Staphylococcus haemolyticus, Streptococcus agalactiae, Streptococcus equi, Streptococcus iniae, Streptococcus uberis, Vibrio alginolyticus, Vibrio anguillarum, Vibrio cholera, Vibrio damsel, Vibrio fluvialis, Vibrio furnissi, Vibrio harveyi, Vibrio hollisae, Vibrio metschnikovii, Vibrio mimicus, Vibrio parahaemolyticus, Vibrio proteolyticis, Vibrio vulnificus, Vibrio splendidus, Yersinia ruckeri and the like.

The term “polypeptide” as used in the context of the invention may refer to a long, continuous, and unbranched peptide and may include cyclic polypeptides. Proteins consist of one or more polypeptides arranged in a biologically functional way and may often be bound to cofactors, or other proteins. In particular, the protein according to any aspect of the present invention may be naturally occurring, de novo and/or synthetic.

The terms “subject”, “patient” and “individual” are used interchangeably and are used in the context of the invention refers to either a human or a non-human animal. These terms include mammals, such as humans, primates, livestock animals (including bovines, porcines, etc.), companion animals (e.g. canines, felines, etc) and rodents (e.g. mice and rats). In particular, the subject is an aquatic animal. The aquatic animal can be any animal, either vertebrate or invertebrate, which lives in the water for most or all of its life. The aquatic animal may be an arthropod for example a Horseshoe crab. In particular, the aquatic animal can be any crustacean which includes but is not limited to crabs, lobsters, crayfish, langoustine, shrimp, and prawn. For example, a prawn can be decapod crustaceans. The term “prawn” can include cold water prawn, warm water prawn, caridean shrimp, whiteleg shrimp, Atlantic white shrimp, Indian prawn, banana prawn, tiger prawn and the like. In another example, the aquatic animal can be any fish, such as, for example, the Toad fish, zebra fish, Grouper or salmon; any crustacean such as, for example fiddler crab, or crayfish; or any cephalopod such as, for example, a squid. The aquatic animal can also be an amphibian such as, for example, a frog or salamander. The aquatic animal can be an animal adapted to fresh water, seawater, or brackish water. Both brackish water and seawater are saltwater. Brackish water has more salinity than fresh water, but less than seawater, such as the water in estuaries.

The term “variant”, as used in the context of the invention can alternatively or additionally be characterised by a certain degree of sequence identity to the parent polypeptide from which it is derived. More precisely, a variant in the context of the present invention exhibits at least 30% sequence identity, in particular at least 40%, 50%, 60%, 70%, 80% or 90% sequence identity. More in particular, a variant in the context of the present invention exhibits at least 95% sequence identity to its parent polypeptide. The variants of the present invention exhibit the indicated sequence identity, and preferably the sequence identity is over a continuous stretch of 100, 150, 200, 300, 315, 320, 330, 340, 344 or more amino acids. The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL available e.g. on http://www.ebi.ac.uk/Tools/clustalw/. Preferred parameters used are the default parameters as they are set on http://www.ebi.ac.uk/Tools/clustalw/ or http://www.ebi.ac.uk/Tools/clustalw2/index.html. The grade of sequence identity (sequence matching) may be calculated using e.g. BLAST, BLAT or BlastZ (or BlastX). Preferably, sequence matching analysis may be supplemented by established homology mapping techniques like Shuffle-LAGAN (Brudno M., Bioinformatics 2003b, 19 Suppl 1 :154-162) or Markov random fields. When percentages of sequence identity are referred to in the present application, these percentages are calculated in relation to the full length of the longer sequence, if not specifically indicated otherwise.

The phrase “Specific pathogen free (SPF) animal” is a special stock of animals that are kept in specific pathogen free facilities under rigorous monitoring system, which are subjected to sensitive and accurate diagnostic methods. The traditional methods of producing SPF includes the animals being repeatedly bred under controlled conditions to maintain their freedom from specific pathogens and the SPF designation itself is tested on a regular basis over an extended period of time. The SPF animals may not innately be resistant to the specified pathogens or infections, although they can possibly be developed as specific pathogen resistant (SPR) species. They are not produced to provide either superior genetic stock or improved culturing attributes such as faster growth. However, these characteristics can be incorporated into SPF stock to increase their commercial value. The SPF status of stock animals may be lost once the animals are removed from the designated facility even if the animals are not infected or develop any other disease symptoms. The SPF animals may be referred to as “high health” stock once they are transferred to other well-established unit with history of disease surveillance.

A person skilled in the art will appreciate that the present invention may be practiced without undue experimentation according to the method given herein. The methods, techniques and chemicals are as described in the references given or from protocols in standard biotechnology and molecular biology textbooks.

In one aspect of the present invention, there is provided a method of producing at least one specific pathogen free (SPF) non-human animal, the method comprising:
(a) selecting a surviving animal in an environment comprising at least one pathogen that is capable of infecting and/or killing the animal;
(b) administration of a fusion protein to the surviving animal wherein the fusion protein comprises at least one polypeptide B which is a Type 1 Ribosome Inactivating Protein (RIP) or fragment thereof; and
(i) at least one polypeptide A which is an Antimicrobial peptide; and/or
(ii) at least one polypeptide C which is a Cationic Antimicrobial Peptide (CAP) or fragment thereof; and
(c) resulting surviving animal is the SPF non-human animal.

In particular, the specific pathogen free non-human animal may be considered an “instant specific pathogen free” or ISPF non-human animal that may be breeding stock indicating that a “viral clean-up” is possible.

The “surviving animal” in step (a) may be any animal that may be capable of enduring the environment with at least one pathogen thus staying alive in the presence of the pathogen. The environment may be considered “challenging” allowing selective breeding to take place thus the surviving animal may be considered a suitable candidate for SPF and/or SPR.

The method may further comprise a step of confirming that the surviving animal from step (a) expresses at least one marker of a pathogen resistant gene before the administration of the fusion protein of step (b). These markers may be well known in the art to be specific the particular animal. In particular, these markers may be known in the art to be expressed in a particular animal that is resistant to at least one pathogen. For example, if the animal is a prawn, the marker may be selected from the group consisting of pmAV, c-type lectin, haemocyanin, beta-integrin, syntenin, alpha-2-macroglobulin, LPS-binding protein, beta-glucan binding protein, catalase gene, Ras-related nuclear protein, caspace-3 like gene, calreticulin, Rab GTPase gene, Mg-SOD gene and the like. Similarly, each species of animal may have markers that are specific to that animal.

For example, the development of WSSV-resistant (WSSV-SPR) lines of P. vannamei is recognized a possibility and because WSSV remains the biggest disease problem in Asian shrimp culture, this would provide a much-needed impetus for the Asian shrimp culture industry as a whole. The recent applications of quantitative genetics to shrimp breeding, including the identification of various molecular markers (particularly microsatellites) associated with disease resistance and growth, offer a method through which the selection of fast-growing, disease resistant strains might soon become much more efficient. It may also shed some light on invertebrate antiviral immunity, about which currently nothing is known. Such disease related markers have already been identified for IHHNV in P. stylirostris (Hizer S. E. et. al., 2002). The genes that are up-regulated in shrimp during a WSSV infection has been reviewed (Liu H. et. al., 2009) and these may now be used as disease resistance markers for selective breeding.

The surviving animal in step (a) may be at least one animal that has been selectively bred for growth prior to carrying out the method according to any aspect of the present invention. In one example, the animals bred in the environment comprising at least one pathogen that is capable of infecting and/or killing the animal have been pre-selected for growth and/or any other advantageous trait and may grow at a faster rate than the wild type of the animal. This may also the SPF animal using any method of the present invention may be achieved earlier than the methods known in the art.

There may be a negative correlation between growth and disease resistance (Argue B. et. al., 2002) and hence, it may be best to begin from a population that had been pre-selected for growth.

The presence of the SPF non-human animal in step (c) may be confirmed using any method known in the art. In particular, the presence of the SPF non-human animal may be confirmed by determining the presence or absence of the virus in the animal. The method of determining may be any method known in the art that is capable of identifying the presence of any genetic material of the virus in the animal. For example, the method of determining the presence of at least one SPF non-human animal may be selected from the group consisting of PCR, ELISA, RT-PCR, LAMP and the like.

Thus, for example, any shrimp candidate or any aquatic animal, may be fed any fusion protein according to any aspect of the present invention along with its feed until it may became PCR negative for the specific viruses to be checked. If this animal were from a known SPF-line that had been selected for growth, preferably over 3 or more generations, and if this animal were to be grown in pond conditions where viruses were common, the ‘survivors’ of any resulting epizootic if any, would have a high probability of carrying resistance genes that may be screened using PCR, RT-PCR or microarrays and these could be used to selectively breed a SPR line over time. Microarrays may be used to study shrimp immune responses under various conditions which is a convenient and rapid method to screen survivor populations for resistance genes.

The SPF animal may free from at least one pathogen selected from the group consisting of cytomegalovirus (CMV), Epstein-Barr virus (EBV), varicella zoster virus (VZV), HSV-1, HSV-2, HSV-6, BK-virus, influenza viruses, respiratory syncytial virus (RSV); human immunodeficiency virus (HIV), hepatitis A, B or C (HBV), polio viruses, enteroviruses, human coxsackie viruses, rhinoviruses, echoviruses, equine encephalitis viruses, rubella viruses, dengue viruses, encephalitis viruses, yellow fever, coronaviruses, vesicular stomatitis viruses, rabies viruses, ebola viruses, parainfluenza viruses, mumps virus, measles virus, respiratory syncytial virus, Hantaan viruses, bunga viruses, phleboviruses and Nairo viruses, hemorrhagic fever viruses, reoviruses, orbiviurses and rotaviruses, parvoviruses, papilloma viruses, polyoma viruses, adenoviruses, herpes simplex virus (HSV) 1 and HSV-2, varicella zoster virus, variola viruses, vaccinia viruses, pox viruses, African swine fever virus, WSSV, HPV, MBV, IHHNV, YHV, TSV, GAV, LSNV, IMNV, MoV, KHV1, KHV2, KHV3, VNN, pancreatic necrosis virus (IPNV), channel catfish virus (CCV), fish lymphocystis disease virus (FLDV), hematopoietic necrosis virus (IHNV) and viral hemorrhagic septicemia virus (VHSV), AVG, AMAV, swine hepatitis E virus, Circoviruses, Herpesviruses, Porcine cytomegalovirus, pseudorabies virus, Feline Panleukopenia virus (FPV), Feline herpesvirus, Feline calicivirus, Feline Leukemia Virus (FeLV), Feline Immunodeficiency Virus (FIV), Rabies virus, canine parvovirus, canine coronavirus, canine distemper virus, canine influenza, canine hepatitis virus, canine herpesvirus, a virus that causes pseudorabies, and canine minute virus.

The fusion protein according to any aspect of the present invention may be an antiviral compound capable of a broad spectrum of applications and that may be economically produced without any limitation of raw material supply unlike certain antiviral compounds known in the art.

In order to achieve broad-spectrum activity, the fusion peptide according to any aspect of the present invention may be able to interfere with viral growth or proliferation in a number of different pathways. The fusion protein may thus have a multifunctional ability. An entire new class of antiviral drugs may thus be produced from the fusion protein according to any aspect of the present invention. The number of combinations and permutations that may be obtained from expressed polypeptides A, B, and C as fusion antiviral proteins potentially numbers in the tens of thousands.

In particular, the fusion protein may comprise at least one formula selected from the group consisting of formulas I-XIX:

A-B-C, Formula I

A-B-C-C, Formula II

A-B, Formula III

A-C-B, Formula IV

C-A-B, Formula V

C-B-A, Formula VI

C-B, Formula VII

B-A-C, Formula VIII

B-A-C-C, Formula IX

B-C-A, Formula X

B-C, Formula XI

B-A, Formula XII

C-C-B-C-C, Formula XIII

C-B-C, Formula XIV

Polypeptide A may be an antimicrobial peptide. In particular, polypeptide A may be a viral entry inhibitory protein. More in particular, polypeptide A may be a defensin, an analogue, or a fragment thereof. Even more in particular, the defensin may be an alpha, a beta, theta defensin, and a member of the Big defensins protein family, an analogue, or a fragment thereof. Polypeptide B may be Type 1 RIP, or a fragment thereof, polypeptide C may be Cationic Antimicrobial Peptide (CAP) or a fragment thereof; and—may be a direct linkage or a linker peptide.

In particular, the linker peptide may comprise a polypeptide sequence: [VPXVG]ₙ,(SEQ ID NO:3) wherein X is an unknown or other amino acid and n is the number of repeats of SEQ ID NO:3 in each linker peptide. For example, n may be 1, 2, 3, 4 or 5. More in particular, X in SEQ ID NO:3 is G and n is 2.

In another example, the linker peptide may be a glycine-serine linker. In particular, the glycine-serine linker may have a sequence of [G-G-G-S]ₙ (SEQ ID NO:27).

In particular, the fusion protein may comprise the formula I:

A-B-C-

wherein, polypeptide A is a defensin (α, β, θ) an analogue, or a fragment thereof. In particular, polypeptide A may be a theta defensin, an analogue, or a fragment thereof, polypeptide B may be Type 1 RIP, or a fragment thereof, and polypeptide C may be CAP, or a fragment thereof and “-” may be a direct linkage or a linker peptide.

More in particular, polypeptide A may be fused to polypeptide B via at least one first linker peptide of SEQ ID NO: 3. Even more in particular, polypeptide A may be fused to polypeptide B via a peptide of SEQ ID NO: 3, wherein X is G and n is 2. Polypeptide B may be directly linked to polypeptide C with no linker peptide in-between. Polypeptide C in formula I may comprise a second linker peptide on the free end not linked to B. The second linker peptide may comprise the formula SEQ ID NO: 3. Even more in particular, in the second linker peptide X is G and n is 2.

Polypeptide A may be a viral entry inhibitor protein. In particular, polypeptide A may be a defensin (α, β, θ) an analogue, or a fragment thereof. In particular, polypeptide A may be a theta defensin of a vertebrate or invertebrate origin. In particular, theta Defensin may be from a bacterium, fungus, mammal, amphibian or reptile. The mammal may be a non-human primate and/or the invertebrate may be a horseshoe crab and/or an insect. The theta Defensin may be selected from the group consisting of Rhesus minidefensin (RTD-1), RTD-2, RTD-3, Retrocyclin-1, Retrocyclin-2, Retrocyclin-3 from Macaca mulatta of SEQ ID Nos: 7-12 respectively and the like (Tang Y Q, 1999; Leonava L, 2001; Wang W, 2004).

The theta Defensin may be synthetic and may be selected from a group of retrocyclin congeners RC100-RC108 and RC110-RC114 of SEQ ID NO:13-25 respectively (Cole et. al. 2002: PNAS, V99(4):1813-1818 ; Wang et. al. 2003: J. Immunol. 170:4708-4716). The sequences of Retrocyclin (RC) 100-108 and RC110-RC114 are shown in Table 1a below.

TABLE 1A
Polypeptide sequences of naturally occurring
and synthetic theta Defensin proteins.

| SEQ ID NO: | Sequences |
|---|---|
| 7 | GFCRCLCRRGVCRCICTR |
| 8 | RCLCRRGVCRCLCRRGVC |
| 9 | RCICTRGFCRCICTRGFC |
| 10 | GICRCICGRGICRCICGR |
| 11 | GICRCICGRGICRCICGR |
| 12 | RICRCICGRRICRCICGR |
| 13 | GICRCICGRGICRCICGR |
| 14 | GICRCICGKGICRCICGR |
| 15 | GICRCYCGRGICRCICGR |
| 16 | GICRCICGRGICRCYCGR |
| 17 | GYCRCICGRGICRCICGR |
| 18 | GICRCICGRGYCRCICGR |
| 19 | GICYCICGRGICRCICGR |
| 20 | GICICICGYGICRCICGR |
| 21 | GICICICGRGICYCICGR |
| 22 | GICICICGRGICYCICGR |
| 23 | RGCICRCIGRGCICRCIG |
| 24 | RGCICRCIGRGCICRCIG |
| 25 | GICRCICGRGICRCICGR |
| 26 | GICRCICGKGICRCYCGR |

Polypeptide A may be a beta defensin. In particular, polypeptide A may be avian beta defensin (AVBD103).

Alpha defensins for human are HNP 1-4 and Human Defensin 5-6, and alpha defensins of mice, monkeys, rats, rabbits, guinea pigs, hamster, horse, elephant, baboon, hedgehog, horse, chimpanzee, orangutan, macaque and marmoset.

Beta defensins are DEFB 1, DEFB 4A, DEFB 4B, DEFB 103A, DEFB 103B, DEFB 104A, DEFB 1046, DEFB 105A, DEFB 1056, DEFB 106A, DEFB 106B, DEFB 107A, DEFB 107B, DEFB 108B, DEFB108 P1-4, DEFB 109 P1, DEFB 109 P1B, DEFB 109 P2-3, DEFB 110, DEFB 112-119, DEFB 121-136.

Big defensins is a diverse family of antimicrobial peptides. Members of the Big defensins protein family originate from (i) Amphioxus—Branchiostoma florida and Branchiostoma belcheri; (ii) Horseshoecrab—Tachypleus tridentatus; (iii) Mussel—Mytilus galloprovincialis; (iv) Clam—Ruditapes philippinam; and (v) Oyster—Crassostrea gigas.

Polypeptide B may be a Type 1 Ribosome Inactivating Protein selected from the group consisting of Ebulitins, Nigritins, Amarandins, Amaranthus antiviral/RIP, Amaranthin, Atriplex patens RIP, Beta vulgaris RIP, β-vulgin, Celosia cristata RIP, Chenopodium album RIP, CAP30B, Spinacea oleracea RIP, Quinqueginsin, Asparins, Agrostin, Dianthins, DAPs, Dianthus chinensis', Lychnin, Petroglaucin, Petrograndin, Saponaria ocymoides RIP, Vacuolas saporin, Saporins, Vaccaria hispanica RIP, Benincasins, Hispin, Byrodin's, Colocins, Cucumis figarei RIP, Melonin, C. moschata RIP, Cucurmosin, Moschatins, Pepocin, Gynostemmin, Gynostemma pentaphyllum RIP, Gypsophilin, Lagenin, Luffaculin, Luffangulin, Luffin, MORs, Momordin II, Momorcharin's, Momorcochin, Momorcochin-S, Sechiumin, Momorgrosvin, Trichoanguin, Kirilowin, α-trichosanthin, TAP-29, Trichokirin, Trichomislin, Trichosanthin, Karasurin, Trichomaglin, Trichobakin, Crotin, Euserratin Antiviral Protein GAP-31, Gelonin, Hura crepitans RIP, Curcin, Jathropa curcas RIP, Mapalmin, Manutins, α-pisavin, Charibdin, Hyacinthus orientalis RIP, Musarmin, Iris hollandica RIP, Cleroendrum aculeatum RIP, CIPs,), Crip-31, Bouganin, Bougainvilla spectbilis RIP, Bougainvillea×buttiana Antiviral protein 1 (BBAP1), Malic enzymes, MAP-S, pokeweed antiviral proteins (PAP), PD-SI, DP-S2, Dodecandrin, PIP, PIP2, Phytolacca octandra anti-viral proteins, Hordeum vulgare RIPs, Hordeum vulgare sub sp. Vulgare Translational inhibitor II, Secale cereale RIP, Tritin, Zea diploperemis RIPs, Malus×domestica RIP, Momordica Anti-HIV Protein, Gelonium multiflorum, Mirabilis expansa 1, phage MU1, betavulgin (Bvg), curcin 2, saporin 6, Maize RIP (B-32), Tobacco RIP (TRIP), Beetins, Mirabilis antiviral protein (MAP), Trichosanthin (TCS), luffins, Momorcharins, Ocymoidin, Bryodin, Pepopsin, 13-trichosanthin, Camphorin, YLP, Insularin, Barley RIP, Tritins, Lamjarin, Volvariella volvacea RIP and the like of plant origin.

Polypeptide C may be selected from the group consisting of Cyclotides, Siamycins, NP-06, Gramicidin A, Circulins, Kalatas, Ginkbilobin, Alpha-Basrubin, Lunatusin, Sesquin, Tricyclon A, Cycloviolacins, Polyphemusins, hfl-B5, Protegrins (Pig Cathelicidin), Rat Defensins, Human β-defensins, Temporins, Caerins, Ranatuerins, Reptile Defensin, Piscidin's, Lactoferricin B, Rabbit Neutrophils, Rabbit α-Defensin, Retrocyclins, Human α-Defensins, Human β-defensin III (HBD3), Rhesus minidefensin (RTD-1,θ-defensin), rhesus θ-defensins, Human neutrophil peptides, Cecropin As, Melittin, EP5-1, Magainin 2s, hybrid (CE-MA), hepcidin TH1-5, Epinecidin-1, Indolicidin, Cathelicidin-4, LL-37 Cathelicidin, Dermaseptins, Maximins, Brevinins, Ranatuerins, Esculentins, Maculatin 1.3, Maximin H5 and Piscidins, Mundticin KS Enterocin CRL-35, Lunatusin, FK-13 (GI-20 is a derivative), Tachyplesins, Alpha-MSH, Antiviral protein Y3, Palustrin-3AR, Ponericin L2, Spinigerin, Melectin, Clavanin B, Cow cathelicidin's, Guinea pig cathelicidin CAP11, Sakacin 5X, Plectasin, Fungal Defensin, GLK-19, lactoferrin (Lf) peptide 2, Alloferon 1, Uperin 3.6, Dahlein 5.6, Ascaphin-8, Human Histatin 5, Guineapig neutrophils, Mytilins, EP5-1,Hexapeptide (synthetic) Corticostatin IV Rabbit Neutrophil 2, Aureins, Latarcin, Plectasin, Cycloviolins, Vary Peptide E, Palicourein, VHL-1, Gaegurin 5, Gaegurin 6 and the like (U.S. Pat. No. 8,076,284 B2; Kim, S. et al, Peptides, 2003, 24, 945-953).

In particular, polypeptide C may be Gaegurin 5, Gaegurin 6, their analogues, derivatives or fragments thereof, which may have pro-apoptotic properties that may act upon drug sensitive and multidrug resistant tumour cell lines.

A Cationic Antimicrobial Peptide (CAP) may be an anti-microbial CAP that may have anticancer and/or antiviral properties. CAPs may be a maximum of 100 amino acids in length. CAPs may either be a naturally occurring CAP with sequence with reported anticancer properties or a synthetic CAP sequence with anticancer properties. CAPs may mostly be of animal origin. However, there may also be CAPs, which are from plants, which include but are not limited to cyclotides. For example, bacteria CAPs may include but are not limited to Siamycin, NP-06 and Gramicidin A. Plant CAPs may include Circulin A, B, Kalata B1 and B8; Plant CAPs which may function as entry inhibitors may include Kalata B8, Ginkbilobin, Alpha-Basrubin, Lunatusin and Sesquin, Circulin A, C and D, Tricyclon A and Cycloviolacin H4. Animal CAPs may include Polyphemusin I and II, hfl-B5, Protegrin (Pig Cathelicidin), Rat Defensin NP1, NP2, NP3 and NP4, Human β-defensin I and II, Temporin A, Temporin-LTc, Temporin-Pta, Caerin 1.1, Ranatuerin 6 and 9, Reptile Defensin and Piscidin 1 and 2, Lactoferricin B, Rabbit Neutrophil-1 Corticostatin III a, Rabbit Neutrophil-3A, Rabbit α-Defensin, Retrocyclin-1, Retrocyclin-2, Retrocyclin-3, Human α-Defensin HNP-1, 2, 3,4,5 & 6, Human β-defensin III (HBD3), Rhesus minidefensin (RTD-1,θ-defensin), RTD-2 rhesus θ-defensin, RTD-3 rhesus θ-defensin, Human neutrophil peptide-2, Human neutrophil peptide-3 and human neutrophil peptide-4, Cecropin A, Melittin, EP5-1, Magainin 2, hepcidin TH1-5, and Epinecidin-1, Indolicidin, Cathelicidin-4,

Human neutrophil peptide-1, LL-37 Cathelicidin, Dermaseptin-S1, S4 and S9, Maximin 1, 3, 4 and 5, Brevinin 1, Ranatuerin 2P, 6 and 9 Esculentin 2P, Esculentin-1 Arb, Caerin 1.1, 1.9 and 4.1, Brevinin-2-related, Maculatin 1.3, Maximin H5 and Piscidin 1 and 2. Other CAPs may include Mundticin KS Enterocin CRL-35, Lunatusin, FK-13 (GI-20 is a derivative), Tachyplesin I, Alpha-MSH, Antiviral protein Y3, Piscidin 3, Palustrin-3AR, Ponericin L2, Spinigerin, Melectin, Clavanin B, Cow cathelicidin BMAP-27, BMAP-28, Guinea pig cathelicidin CAP11, Sakacin 5X, Plectasin, Fungal Defensin, GLK-19, lactoferrin (Lf) peptide 2, Kalata B8, Tricyclon A, Alloferon 1, Uperin 3.6, Dahlein 5.6, Ascaphin-8, Human Histatin 5, Guineapig neutrophil CAP2 & CAP1, Mytilin B & C, EP5-1, and Hexapeptide (synthetic) Corticostatin IV Rabbit Neutrophil 2.

The Type 1 RIP may:
(i) act as a pro-apoptotic polypeptide which up regulate pro-apoptotic genes that may include but not limited to caspase-12, Bax and the like, or down regulate anti-apoptotic gene including but not limited to BcI-2 and the like in tumour or cancer cells (Fan, J-M., et al, Mol Biotechnol, 2008, 39, 79-86);
(ii) act as a DNA glycosylase/apurinic (AP) lyase capable of irreversibly relaxing tumour or cancer cell supercoiled DNA and catalyzing double-stranded breakage to form inactive products;
(iii) act in alternative cytochrome pathways as well as Mn²⁺ and Zn²⁺ interactions with negatively charged surfaces next to catalytic sites, facilitating DNA substrate binding instead of directly participating in catalysis (Wang et al, Cell, 1999, 99, 433-442);
(iv) as an RNA N-Glycosidase which hydrolyses the N-C glycosidic bond of adenosine at position 4324 of the universally conserved sarcin/ricin domain(S/R domain) of the 28S-rRNA in the eukaryotic ribosome and render it incapable of carrying out protein synthesis thus, functionally, blocking translation.

In particular, the Type 1 RIP may be selected from the group consisting of α-Ebulitin, β-Ebulitin, γ-Ebulitin, Nigritin f1, Nigritin f2, Amarandin-S, Amaranthus antiviral/RIP, Amarandin-1, Amarandin-2, Amaranthin, Atriplex patens RIP, Beta vulgaris RIP, β-vulgin, Celosia cristata RIP, Chenopodium album RIP, CAP30B, Spinacea oleracea RIP, Quinqueginsin, Asparin 1, Asparin 2, Agrostin, Dianthin 29, DAP-30, DAP-32, Dianthin 30, Dianthus chinensis RIP1, Dianthus chinensis RIP2, Dianthus chinensis RIP3, Lychnin, Petroglaucin, Petrograndin, Saponaria ocymoides RIP, Vacuolas saporin, Saporin-1, Saporin-2, Saporin-3, Saporin-5, Saporin-6, Saporin-7, Saporin-9, Vaccaria hispanica RIP, Benincasin, α-benincasin, β-benincasin, Hispin, Byrodin I, Byrodin II, Colocin I, Colocin 2, Cucumis figarei RIP, Melonin, C. moschata RIP, Cucurmosin, Moschatin, Moschatin I, Moschatin II, Moschatin III, Moschatin IV, Moschatin V, Pepocin, Gynostemmin I, Gynostemmin II, Gynostemmin III, Gynostemmin IV, Gynostemmin V, Gynostemma pentaphyllum RIP, Gypsophilin, Lagenin, Luffaculin, Luffangulin, Luffin-alpha, Luffin-B, MOR-I, MOR-II, Momordin II, Alpha-momorcharin, p-momorcharin, γδ-momorcharin, γ-momorcharin, Momorcochin, Momorcochin-S, Sechiumin, Momorgrosvin, Trichoanguin, α-kirilowin, β-kirilowin, α-trichosanthin, TAP-29, Trichokirin, Trichomislin, Trichosanthin, Karasurin-A, Karasurin-B, Trichomaglin, Trichobakin, Crotin 2, Crotin 3, Euserratin 1, Euserratin 2, Antiviral Protein GAP-31, Gelonin, Hura crepitans RIP, Curcin, Jathropa curcas RIP, Mapalmin, Manutin 1, Manutin 2, α-pisavin, Charibdin, Hyacinthus orientalis RIP, Musarmin 1, Musarmin 2, Musarmin 3, Musarmin 4, Iris hollandica RIP, Cleroendrum aculeatum RIP, CIP-29, CIP-34, Crip-31, Bouganin, Bougainvilla spectbilis RIP, Bougainvillea×buttiana Antiviral protein 1 (BBAP1), malic enzyme 1 (ME1), ME2, MAP-S, pokeweed antiviral protein (PAPa-1), PAPa-2, PAP-alpha, PAP-I, PAP-II, PAP-S, PD-SI, DP-S2, Dodecandrin, Anti-viral protein PAP, PIP, PIP2, Phytolacca octandra anti-viral protein, Phytolacca, octandra anti-viral protein II, Hordeum vulgare RIP-I, Hordeum vulgare RIP-II, Hordeum vulgare sub sp. Vulgare Translational inhibitor II, Secale cereale RIP, Tritin, Zea, diploperemis RIP-I, Zea diploperemis RIP-II, Malus×domestica RIP, Momordica Anti-HIV Protein (MAP30), Gelonium multiflorum (GAP31), pokeweed antiviral protein (PAP), Mirabilis expansa 1 (ME1), malic enzyme 2 (ME2), Bougainvillea×buttiana antiviral protein 1 (BBAP1), phage MU1, betavulgin (Bvg), curcin 2, saporin 6, Maize RIP (B-32), Tobacco RIP (TRIP), beetin (BE), BE27, Mirabilis antiviral protein (MAP), Trichosanthin (TCS), α-luffin, α-Momorcharin (α-MMC), β-MMC luffin, Ocymoidin, Bryodin, Pepopsin, β-trichosanthin, Camphorin, YLP, Insularin, Barley RIP, Tritins, Lamjarin, Volvarielia volvacea RIP and the like of plant origin.

In particular, polypeptide A may be a Retrocyclin, polypeptide B may be MAP30 and polypeptide C may be a Dermaseptin. More in particular, polypeptide A may be Retrocyclin 101 (RC101) and polypeptide C may be Dermaseptin 1. A polypeptide comprising RC101, MAP30 and Dermaseptin 1 as polypeptide A, B and C respectively is termed RetroMAD1 in the present invention.

In particular, polypeptide A may comprise amino acid sequence with SEQ ID NO: 4, a fragment or variant thereof, polypeptide B may comprise amino acid sequence with SEQ ID NO:5, a fragment or variant thereof, and polypeptide C may comprise amino acid sequence with SEQ ID NO:6, a fragment or variant thereof.

More in particular, the fusion protein according to any aspect of the present invention may comprise the amino acid sequence SEQ ID NO:1. The fusion protein or the basic unit of the fusion protein may have a molecular weight of about 30-50 kDa. In particular, the molecular weight of the fusion protein may be 31, 32, 33, 34, 35, 36, 37, 38, 39, 40, 41, 42, 43, 44, 36.5, 37, 37.5, 37.8, 38, 39, 40, 41, 42, 43, 44, 45, 46, 47, 48 or 49 kDa. The fusion protein may comprise repeats of the basic unit. A skilled person would understand that the weight of the fusion protein would be dependent on the multiples of the basic unit present in the protein. The nucleic acid coding for the fusion protein of SEQ ID NO:1 may be found in SEQ ID NO:2. The sequences are provided in Table 1b below.

In particular, polypeptide B may be Type 1 RIP, or a fragment thereof, and polypeptide C may be Cationic Antimicrobial Peptide or a fragment thereof; and—may be a direct linkage or a linker peptide.

In one example, polypeptide A may be Avian β-Defensin 103 (AVBD103), polypeptide B may be MAP30 and polypeptide C may be Mytilin C10C. In another example, the fusion protein may comprise the formula XIV:

C-B-C

TABLE 1b
Sequences of polypeptides and polynucleotides of the present invention.

| SEQ | |
|---|---|
| ID | |
| NO. | Sequences |
| 1 | MKYLLPTAAAGLLLLAAQPAMAMGRICRCICGRGICRCICGVPGVGVPGVGGATGSDVNFDLSTATAKTY |
| | TKFIEDFRATLPFSHKVYDIPLLYSTISDSRRFILLDLTSYAYETISVAIDVTNVYVVAYRTRDVSYFFK |
| | ESPPEAYNILFKGTRKITLPYTGNYENLQTAAHKIRENIDLGLPALSSAITTLFYYNAQSAPSALLVLIQ |
| | TTAEAARFKYIERHVAKYVATNFKPNLAIISLENQWSALSKQIFLAQNQGGKFRNPVDLIKPTGERFQVT |
| | NVDSDVVKGNIKLLLNSRASTADENFITTMTLLGESVVEFPWALWKTMLKELGTMALHAGKAALGAAADT |
| | ISQGTQVPGVGVPGVGKLAAALEHHHHHH |
| 2 | atgaaatacctgctgccgaccgctgctgctggtctgctgctcctcgctgcccagccggcgatggccatgg |
| | ggcgtatttgccgttgcatttgcggccgtggcatttgccgctgcatctgtggcgtgccgggtgttggtgt |
| | tccgggtgtgggtggtgcgaccggatccgatgtgaactttgatctgagcaccgcgaccgcgaaaacctat |
| | accaaattcatcgaagattttcgtgcgaccctgccgtttagccataaagtgtatgatatcccgctgctgt |
| | atagcaccattagcgatagccgtcgttttattctgctggatctgaccagctatgcgtatgaaaccattag |
| | cgtggcgattgatgtgaccaacgtgtatgtggtggcgtatcgtacccgtgatgtgagctactttttcaaa |
| | gaaagcccgccggaagcgtacaacattctgtttaaaggcacccgtaaaattaccctgccgtataccggca |
| | actatgaaaacctgcagaccgcggcgcataaaattcgtgaaaacatcgatctgggcctgccggccctgag |
| | cagcgcgattaccaccctgttttattataacgcgcagagcgcgccgagcgcgctgctggtgctgattcag |
| | accaccgcggaagcggcgcgttttaaatatattgaacgccacgtggcgaaatatgtggcgaccaacttta |
| | aaccgaacctggccattattagcctggaaaaccagtggagcgccctgagcaaacaaatttttctggccca |
| | gaaccagggcggcaaatttcgtaatccggtggatctgattaaaccgaccggcgaacgttttcaggtgacc |
| | aatgtggatagcgatgtggtgaaaggcaacattaaactgctgctgaacagccgtgcgagcaccgcggatg |
| | aaaactttattaccaccatgaccctgctgggcgaaagcgtggtggaattcccgtgggcgctgtggaaaac |
| | catgctgaaagaactgggcaccatggcgctgcatgcgggtaaagcggcgctgggtgcggcagcggatacc |
| | attagccagggcacccaggttccgggcgtgggcgttccgggcgttggtaagcttgcggccgcactcgagc |
| | accaccaccaccaccactga |
| 3 | [VPXVG]ₙ |
| 4 | GRICRCICGRGICRCICG |
| 5 | GSDVNFDLSTATAKTYTKFIEDFRATLPFSHKVYDIPLLYSTISDSRRFILLDLTSYAYETISVAIDVTN |
| | VYVVAYRTRDVSYFFKESPPEAYNILFKGTRKITLPYTGNYENLQTAAHKIRENIDLGLPALSSAITTLF |
| | YYNAQSAPSALLVLIQTTAEAARFKYIERHVAKYVATNFKPNLAIISLENQWSALSKQIFLAQNQGGKFR |
| | NPVDLIKPTGERFQVTNVDSDVVKGNIKLLLNSRASTADENFITTMTLLGESVVEFPW |
| 6 | ALWKTMLKELGTMALHAGKAALGAAADTISQGTQ |

In another example, the fusion protein may be Amatilin, RetroGAD1, Tamapal1 and the like. DNA and polypeptide sequences of Amatilin, RetroGAD1, and Tamapal1 are presented in Tables 1d and 1e.

Investigations on thermal behaviour of drug samples are important for obtaining information for their processing in pharmaceutical industry, for predicting their shelf lives and also for suitable storage conditions. These drugs were shown to be thermally stable.

TABLE 1c
The polypeptides used for each drug.

| Example | Polypeptide A | Polypeptide B | Polypeptide C |
|---|---|---|---|
| Fusion peptide | Defensin | RIP | CAP |
| RetroMAD1 | Retrocyclin 101 | MAP30 | Dermaseptin1 |
| RetroGAD1 | Retrocyclin 101 | GAP31 | Dermaseptin1 |
| Tamapal1 | Tachyplesin | MAP30 | Alloferon1 |
| Amatilin | AVBD103 | MAP30 | Mytillin C10C |

TABLE 1d
DNA sequences of Amatilin, RetroGAD1 and Tamapal1

| | SEQ | |
|---|---|---|
| Fusion | ID | |
| Protein | NO. | DNA Sequence |
| Amatilin | 37 | GGGCAGTGAGCGGAAGGCCCATGAGGCCAGTTAATTAAGAGGTACCGAATTCTCAT |
| | | TCGGTTTGTGTAGATTGAGAAGAGGTTTCTGTGCTCACGGTAGATGTAGATTCCCA |
| | | TCCATCCCAATCGGTAGATGTTCCAGATTCGTTCAGTGTTGTAGAAGAGTTTGGGT |
| | | CCCAGGTGTTGGTGTTCCAGGTGTTGGAGGTGCTACTGGTTCTGATGTTAACTTCG |
| | | ACTTGTCCACTGCTACTGCTAAGACTTACACTAAGTTCATCGAGGACTTCAGAGCT |
| | | ACTTTGCCATTCTCCCACAAGGTTTACGACATCCCTTTGTTGTACTCCACTATCTC |
| | | CGACTCCAGAAGATTCATCTTGTTGAACTTGACTTCCTACGCTTACGAGACTATCT |
| | | CCGTTGCTATCGACGTTACAAACGTTTACGTTGTTGCTTACAGAACTAGAGATGTT |
| | | TCCTACTTCTTCAAAGAGTCCCCACCAGAGGCTTACAACATCTTGTTCAAGGGTAC |
| | | TAGAAAGATCACTTTGCCATACACTGGTAACTACGAGAACTTGCAGACTGCTGCTC |
| | | ACAAGATCAGAGAGAACATCGACTTGGGTTTGCCAGCTTTGTCCTCCGCTATCACT |
| | | ACTTTGTTCTACTACAACGCTCAGTCCGCTCCATCCGCTTTGTTGGTTTTGATCCA |
| | | GACTACTGCTGAGGCTGCTAGATTCAAGTACATCGAGAGACACGTTGCTAAGTACG |
| | | TTGCTACAAACTTCAAGCCAAACTTGGCTATCATCTCCTTGGAGAACCAGTGGTCT |
| | | GCTTTGTCCAAGCAGATCTTCTTGGCTCAAAACCAGGGTGGTAAGTTCAGAAACCC |
| | | AGTCGACTTGATCAAGCCAACCGGTGAGAGATTCCAGGTTACTAATGTTGACTCCG |
| | | ACGTTGTTAAGGGTAACATCAAGTTGTTGTTGAACTCCAGAGCTTCCACTGCTGAC |
| | | GAGAACTTCATCACTACTATGACTTTGTTGGGTGAGTCCGTTGTTAACTCCTGTGC |
| | | TTCCAGATGTAAGGGTCACTGTAGAGCTAGAAGATGTGGTTACTACGTTTCCGTTC |
| | | TGTACAGAGGTAGATGTTACTGTAAATGTTTGAGATGTGTCCCCGGTGTTGGAGTC |
| | | CCTGGTGTCGGTGCGGCCGCGAGCTCATGGCGCGCCTAGGCCTTGACGGCCTTCCG |
| | | CCAATTCGC |
| RetroGAD1 | 38 | CGAATTGGCGGAAGGCCGTCAAGGCCACGTGTCTTGTCCAGGTACCGAATTCGGAA |
| | | TCTGTAGATGCATCTGCGGTAGAGGTATCTGCAGATGTATTTGTGGAAGAGTCCCA |
| | | GGTGTTGGTGTTCCAGGTGTTGGAGGTGCTACTGGTTCTGGTTTGGACACTGTTTC |
| | | ATTCTCCACTAAGGGTGCTACTTACATCACTTACGTTAACTTTTTGAACGAGTTGA |
| | | GAGTTAAGTTGAAGCCAGAGGGTAACTCCCACGGTATCCCTTTGTTGAGAAAGAAG |
| | | TGTGACGACCCAGGTAAGTGTTTCGTTTTGGTTGCTTTGTCCAACGACAACGGTCA |
| | | GTTGGCTGAGATTGCTATCGACGTTACTTCCGTTTACGTTGTTGGTTACCAGGTTA |
| | | GAAACAGATCCTACTTCTTCAAGGACGCTCCAGACGCTGCTTACGAAGGTTTGTTC |
| | | AAGAACACTATCAAGACTAGATTGCACTTCGGTGGTTCCTACCCATCTTTGGAAGG |
| | | TGAGAAGGCTTACAGAGAGACTACTGACTTGGGTATCGAGCCATTGAGAATCGGTA |
| | | TCAAGAAGTTGGACGAGAACGCTATCGACAACTACAAGCCAACTGAGATCGCTTCC |
| | | TCCTTGTTGGTTGTTATCCAGATGGTTTCCGAGGCTGCTAGATTCACTTTCATCGA |
| | | GAACCAGATCAGAAACAACTTCCAGCAGAGAATCAGACCAGCTAACAACACTATTT |
| | | CCTTGGAGAACAAGTGGGGTAAGTTGTCCTTCCAGATCAGAACATCCGGTGCTAAC |
| | | GGTATGTTCTCTGAGGCTGTTGAGTTGGAGAGAGCTAACGGTAAGAAGTACTACGT |
| | | TACTGCTGTTGACCAGGTTAAGCCAAAGATCGCTTTGTTGAAGTTCGTTGACAAGG |
| | | ACCCAAAGGGTTTGTGGTCCAAGATCAAAGAGGCTGCTAAGGCTGCTGGTAAGGCT |
| | | GCTTTGAATGCTGTTACTGGTTTGGTTAACCAGGGTGACCAACCATCTGTCCCTGG |
| | | TGTTGGAGTCCCTGGTGTCGGTGCGGCCGCGAGCTCTGGAGCACAAGACTGGCCTC |
| | | ATGGGCCTTCCGCTCACTGC |
| Tamapal1 | 39 | GGATCCGTTCCGGGTGTGGGTGTTCCGGGTGTTGGTAAATGGTGTTTCGTGTTTGT |
| | | TATCGCGGTATTTGTTATCGTCGTTGTCGTGTGCCAGGCGTTGGCGTTCCAGGCGT |
| | | GGGTGGTGCAACCGGTAGTGATGTTAATTTTGATCTGAGCACCGCAACCGCAAAAA |
| | | CCTATACCAAATTTATCGAAGATTTTCGTGCAACCCTGCCGTTTAGCCATAAAGTT |
| | | TATGATATTCCGCTGCTGTATAGCACCATTAGCGATAGCCGTCGTTTTATTCTGCT |
| | | GAATCTGACCAGCTATGCCTATGAAACCATTAGCGTTGCAATTGATGTGACCAATG |
| | | TTTATGTTGTTGCATATCGTACCCGTGATGTGAGCTATTTTTTCAAAGAAAGCCCT |
| | | CCGGAAGCCTATAACATTCTGTTTAAAGGCACCCGCAAAATCACCCTGCCGTATAC |
| | | CGGTAATTATGAAAATCTGCAGACCGCAGCACATAAAATTCGCGAAAATATTGATC |
| | | TGGGTCTGCCTGCACTGAGCAGCGCAATTACCACCCTGTTTTATTACAATGCACAG |
| | | AGCGCACCGAGCGCACTGCTGGTTCTGATTCAGACCACCGCAGAAGCAGCACGCTT |
| | | TAAATACATTGAACGTCATGTTGCCAAATACGTGGCCACCAACTTTAAACCGAATC |
| | | TGGCAATTATTAGCCTGGAAAATCAGTGGTCAGCACTGAGCAAACAAATTTTTCTG |
| | | GCACAGAATCAGGGTGGCAAATTTCGTAATCCGGTTGATCTGATTAAACCG |
| | | ACCGGTGAACGTTTTCAGGTTACCAATGTTGATAGTGATGTGGTGAAAGGCAACAT |
| | | TAAACTGCTGCTGAATAGCCGTGCAAGCACCGCAGATGAAAACTTTATTACCACCA |
| | | TGACCCTGCTGGGTGAAAGCGTTGTTAATGTTCCTGGTGTTGGCGTGCCTGGTGTT |
| | | GGTCATGGTGTTAGCGGTCATGGTCAGCATGGTGTTCATGGTTAAAAGCTT |

TABLE 1e
Polypeptide sequences of Amatilin, RetroGAD1 and Tamapal1

| | SEQ | |
|---|---|---|
| Fusion | ID | |
| Protein | NO. | Protein Sequence |
| Amatilin | 28 | SFGLCRLRRGFCAHGRCRFPSIPIGRCSRFVQCCRRVWVPGVGVPGVGGATGSDVNF |
| | | DLSTATAKTYTKFIEDFRATLPFSHKVYDIPLLYSTISDSRRFILLNLTSYAYETIS |
| | | VAIDVTNVYVVAYRTRDVSYFFKESPPEAYNILFKGTRKITLPYTGNYENLQTAAHK |
| | | IRENIDLGLPALSSAITTLFYYNAQSAPSALLVLIQTTAEAARFKYIERHVAKYVAT |
| | | NFKPNLAIISLENQWSALSKQIFLAQNQGGKFRNPVDLIKPTGERFQVTNVDSDVVK |
| | | GNIKLLLNSRASTADENFITTMTLLGESVVNSCASRCKGHCRARRCGYYVSVLYRGR |
| | | CYCKCLRCVPGVGVPGVG |
| RetroGAD | 36 | GICRCIGRGICRCICGRVPGVGVPGVGGATGSGLDTVSFSTKGATYITYVNFLNELR |
| 1 | | VKLKPEGNSHGIPLLRKKCDDPGKCFVLVALSNDNGQLAEIAIDVTSVYVVGYQVRN |
| | | RSYFFKDAPDAAYEGLFKNTIKTRLHFGGSYPSLEGEKAYRETTDLGIEPLRIGIKK |
| | | LDENAIDNYKPTEIASSLLVVIQMVSEAARFTFIENQIRNNFQQRIRPANNTISLEN |
| | | KWGKLSFQIRTSGANGMFSEAVELERANGKKYYVTAVDQVKPKIALLKFVDKDPKGL |
| | | WSKIKEAAKAAGKAALNAVTGLVNQGDQPSVPGVGVPGVG |
| Tamapal1 | 34 | VPGVGVPGVGKWCFRVCYRGICYRRCRVPGVGVPGVGGATGSDVNFDLSTATAKTYT |
| | | KFIEDFRATLPFSHKVYDIPLLYSTISDSRRFILLNLTSYAYETISVAIDVTNVYVV |
| | | AYRTRDVSYFFKESPPEAYNILFKGTRKITLPYTGNYENLQTAAHKIRENIDLGLPA |
| | | LSSAITTLFYYNAQSAPSALLVLIQTTAEAARFKYIERHVAKYVATNFKPNLAIISL |
| | | ENQWSALSKQIFLAQNQGGKFRNPVDLIKPTGERFQVTNVDSDVVKGNIKLLLNSRA |
| | | STADENFITTMTLLGESVVNVPGVGVPGVGHGVSGHGQHGVHG |

Modifications and changes may be made in the structure of the peptides of the present invention and DNA segments, which encode them and still obtain a functional molecule that encodes a protein or peptide with desirable characteristics. The amino acids changes may be achieved by changing the codons of the DNA sequence. For example, certain amino acids may be substituted for other amino acids in a protein structure without appreciable loss of interactive binding capacity with structures such as, for example, tumour or cancer cell-binding regions of fusion proteins. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. Various changes may be made in the peptide sequences of the disclosed compositions, or corresponding DNA sequences, which encode said proteins without appreciable loss of their biological utility or activity. Amino acid substitutions of the fusion protein according to the present invention may be possible without affecting the antitumour or anticancer effect of the isolated peptides of the invention, provided that the substitutions provide amino acids having sufficiently similar properties to the ones in the original sequences.

The fusion peptide according to any aspect of the present invention may be thermostable over a prolonged period of time. Thermostability is an industrially significant attribute as cold-chain transportation will greatly increase logistics and handling costs that will contribute to the overall total cost of the medication. Also, if the drug is to be carried about to be consumed before meals, patient compliance will suffer if the requirement of low temperature storage in an absolute necessity. Thus, the ability to remain stable for 7 days even at elevated temperatures will allow for a wider usage and application of the therapeutic protein.

The fusion protein may further comprise a pharmaceutically acceptable carrier, excipient, adjuvant, diluent and/or detergent. Such formulations therefore include, in addition to the fusion protein, a physiologically acceptable carrier or diluent, possibly in admixture with one or more other agents such as other antibodies or drugs, such as an antibiotic. Suitable carriers include, but are not limited to, physiological saline, phosphate buffered saline, phosphate buffered saline glucose and buffered saline. Alternatively, the fusion protein may be lyophilized (freeze dried) and reconstituted for use when needed by the addition of an aqueous buffered solution as described above. Routes of administration are routinely parenteral, including intravenous, intramuscular, subcutaneous and intraperitoneal injection or oral delivery. The administration can be systemic and/or local. The medicament may be used for topical or parenteral administration, such as subcutaneous, intradermal, intraperitoneal, intravenous, intramuscular or oral administration. For this, the fusion protein may be dissolved or suspended in a pharmaceutically acceptable, preferably aqueous carrier. The medicament may contain excipients, such as buffers, binding agents, blasting agents, diluents, flavours, lubricants, etc.

In particular, the fusion protein may be produced as a solid dose by means of Supercritical Fluid Drying (SCFD) used to dry and produce a micronized form of powdered free-flowing RetroMAD1. The powder may be for incorporation into tablets, capsules and animal feed pellets whether for terrestrial or aquatic application. This allows for high process yields and may enable further ease of oral drug delivery in tablet and/or capsule form.

In particular, the fusion protein may be administered orally. In particular, the presence of MAP30 surprisingly renders the fusion protein according to any aspect of the present invention stable for oral administration. In particular, the fusion protein may be administered with or before food. More in particular, when the fusion protein is administered before food, it may be done with a drink for example water. In the case of aquatic animals that do not ‘drink’, it may be effectively administered by top-coating the feed pellets with the fusion protein. Furthermore, they can be coated further with proteins to prevent leaching. A non-limiting example is the use of proteins from chicken eggs and the like to protect against leaching.

The fusion protein according to any aspect of the present invention may be capable of maintaining its form in the digestive tract without fragmentation or enzymatic digestion. In one example, the fusion protein may be in a liquid form. In particular, the fusion protein may be ingested, as a drink diluted with water, or the like, and the retention time in either stomach or duodenum is only a matter of minutes allowing the protein to reach its target point without being digested.

The dosage of the fusion protein according to the present invention to be administered to a non-human animal may vary with the precise nature of the condition being treated and the recipient of the treatment. The dose will generally be in the range of about 0.005 to about 1000 mg for an adult patient, usually administered daily for a period between 1 day to 2 years. In particular, the daily dose may be 0.5 to 100 mg per day. In particular the daily dose may be about 0.8, 1, 1.2, 1.5, 2, 2.5, 3.2, 4, 4.5, 5, 10, 15, 20, 30, 45, 50, 75, 80, 90, 95 mg per day. The dosage may be applied in such a manner that the ligand may be present in the medicament in concentrations that provide in vivo concentrations of said ligand in a patient to be treated of between 0.001 mg/kg/day and 5 mg/kg/day. In one embodiment, the medicament, the peptide or ligand according to the invention is present in an amount to achieve a concentration in vivo of 1 μg/ml or above with a maximum concentration of 100 μg/ml. the dosage regime may be varied depending on the results on the patient.

The fusion protein may be pegylated to aid in the medicament being suitable for oral delivery. In particular, the fusion protein may be pegylated with any PEG known in the art. The PEG may be selected from the group consisting of but not limited to PEG200, 300, 400, 500, 600, 700, 800, 900, 1000, 1100, 1200, 1300, 1400, 1500, 1600, 1700, 1800, 1900, 2000, 2100, 2200, 2300,2400, 2500, 2600, 2700, 2800, 3000, 3250, 3350, 3500, 3750, 4000, 4250, 4500, 4750, 5000, 5500, 6000, 6500, 7000, 7500, 8000 and the like.

SPF animal may be free from only the pathogens that they have been tested for. In shrimp for example, typically this may consist of the viral pathogens which are known to cause major losses to the shrimp culture industry, including WSSV, YHV, TSV, IHHNV, BPV, HPV 3 and the like. However, when new diseases may emerge from mutations of previously non-pathogenic organisms—i.e. the highly mutable RNA viruses the SPF animal may not survive. Hence, it remains a possibility that importation of SPF shrimp may not rule out simultaneous importation of pathogens. Also, if SPF shrimp are stocked into facilities with high viral loads, substantial mortality can result as they are not necessarily more resistant to these diseases than non-SPF shrimp, and in some cases, less so. They may thus be more suited to culture in biosecure systems, which may explain the reliance of the big, non-biosecure pond farms of Latin America on SPR (Specific Pathogen Resistant), rather than SPF shrimp. Accordingly, even though SPF animals have their advantages, they have their limitations and an SPR animal may be needed that may be capable of resistance to all pathogens.

In another aspect of the present invention, there is provided a method of producing at least one specific pathogen resistant (SPR) non-human animal, the method comprising:
(a) producing a specific pathogen free animal according to any method of the present invention; and
(b) selective breeding of a male and female SPF non-human animal to produce a SPR non-human animal offspring.

According to a further aspect of the present invention, there is provided a specific pathogen free or resistant non-human animal produced by any method of the present invention. The animal may be an aquatic animal. More in particular, the aquatic animal may be a prawn of any species or a fish. In one example, the animal may be a non-aquatic animal for example a bird like a chicken and the like. The animal may be of any age and include every stage of the life-cycle of the animal. In particular, the animal may include an egg, larvae and the like of the animal.

A person skilled in the art will appreciate that the present invention may be practised without undue experimentation according to the method given herein. The methods, techniques and chemicals are as described in the references given or from protocols in standard biotechnology and molecular biology text books.

The fusion protein and/or pharmaceutical composition according to any aspect of the present invention may result in no or substantially no toxic side effects when taken by the animal.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Green, Molecular Cloning: A Laboratory Manual, Cold Springs Harbor Laboratory (Fourth Edition), New York (2012).

### Example 1

### Construction and Design of Expression Vector

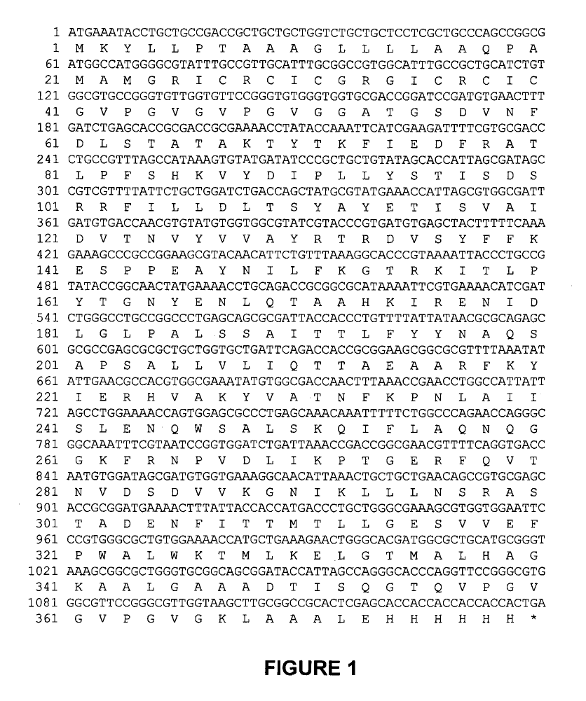

The gene encoding RetroMAD1 A-B-C with SEQ ID NO:1 was synthesized and cloned into backbone of vector pGA4 at the KpnI/SacI site by contract service (GeneArt AG, Germany). The expected product size was 1140 bp, which encoded a 379 amino acid and an expected size of 41.2 kDa. The polynucleotide sequence and the translated polypeptide sequence are shown in FIG. 1 from PCT. The gene was sub-cloned into a pET expression vector (Novagen), pET-26(b) at the NcoI/HindIII sites. Kanamycin was used as a marker for selection and maintenance of culture purposes. This vector was inducible under the addition of isopropyl-beta-D-thiogalactopyranoside (IPTG). The plasmid, pRMD1 was then transformed into BL21(DE23) cells (Novagen) and plated on a selective media with Kanamycin.

Expression of RetroMAD1 from E. coli

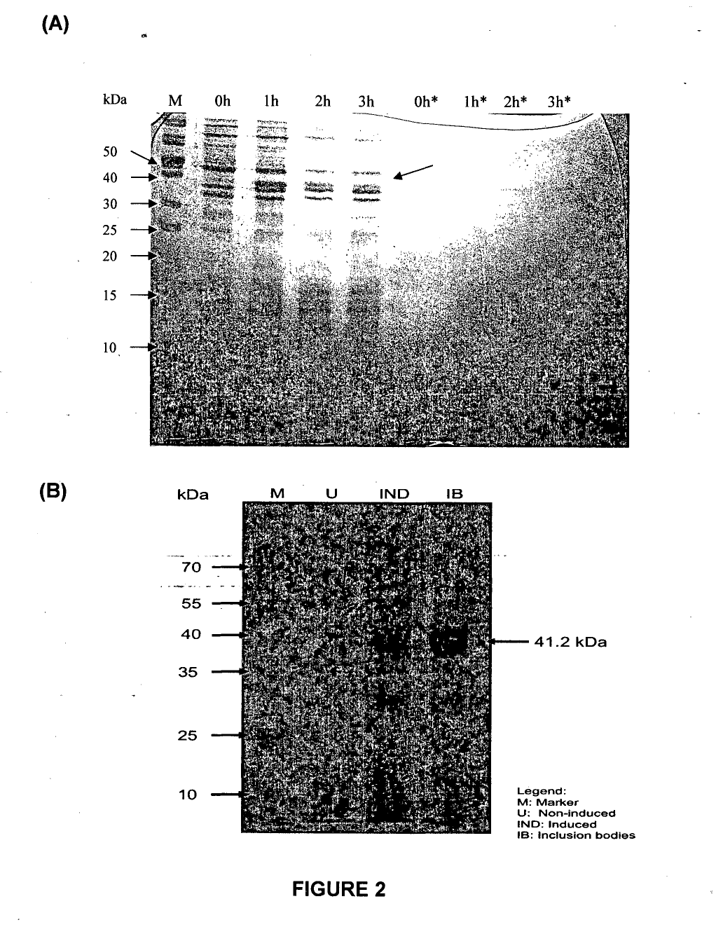

One recombinant clone was grown in 10 ml of LB Bertani (DIFCO) medium, supplemented with 30 μg/ml kanamycin, at 37° C. overnight. This culture was used to inoculate 100 ml of LB Bertani supplemented with 30 μg/ml kanamycin and grown at 37° C. until the optical reading was 0.4-0.6 at 600 nm. IPTG was added at 1.0 mM final concentration. The growth period continued for 3 hours. An SDS-PAGE analysis of the fraction of RetroMAD1 in cells extracted in electrophoresis loading buffer showed that a protein had a molecular mass of about 37.5 kDa, the expected molecular size of RetroMAD1 was produced in the induced cells only (FIG. 2A). Further solubility analysis by SDS-PAGE revealed that RetroMAD1 was found in the pellet fraction and not in the supernatant fraction of the E. coli indicating that the protein was expressed and produced as inclusion bodies as shown in FIG. 2B.

### Isolation and Purification of RetroMAD1

Cells from 100 ml of induced culture were harvested by centrifugation for 10 min at 5000×g at 15° C. The cells were suspended in a lysis buffer containing 20 mM Tris-HCl (pH 7.5), 10 mM EDTA and 1% Triton-X 100. Cells were disrupted by sonication. The insoluble fraction was isolated from the soluble fraction by centrifugation at 8,000×g for 20 min. The supernatant was discarded and the pellet was further washed by repeating the same step. The pellet was further washed twice with RO water by resuspension via sonication and separation by centrifugation.

### Solubilization of RetroMAD1

The insoluble material was dissolved and sonicated in 10 ml of 5-8 Urea or 6M Guanidine Hydrochloride and supplemented with 2-5% of Sodium-lauryl sarcosine and 100 mM β-mercaptoethanol. The solubilisation was carried out overnight. The solubilised protein was separated from the bacterial cell wall by centrifugation at 8,000×g for 20 minutes.

### Refolding of RetroMAD1

Renaturation of the protein was carried out by using dialysis. The protein (10 ml) was dialysed in a 15 kDa molecular weight cut-off dialysis membrane (Spectra/Por Lab). The protein was dialysed in 5 L of RO water with the pH of 11.0 adjusted by NaOH. Incubation was done at room temperature for 15-20 hours. The refolded protein was transferred to a 50 ml tube and centrifuged at 8,000×g to separate any insoluble material. Renatured protein was stored at −20° C. until further use. The bioactivity of RetroMAD1 in the following examples is proof of successful refolding of the protein.

### Example 2

### Elimination of Hepatopancreatic ParvoVirus (HPV) from Shrimp

### Shrimp Culture and RetroMAD1 Treatment

Naturally infected HPV shrimp (150 pieces) was obtained from a local aquarium shop. Twenty pieces of of randomly selected shrimp was selected for DNA extraction to confirm for HPV (Hepatopancreatic Parvo Virus) infection in the population. For the experiment, 56 shrimps were reared in two 20 liters tank (24 each) containing de-chlorinated fresh water equipped with aeration. Water exchange was carried out at 20% every two days. Shrimps were acclimatized for one week before the experiment.

For the experiment both tanks were given 0.25 mg of feed daily, divided into 3 meals. Treated tanks were a given a dose of 25 ug of RetroMAD1 absorbed into the commercial feed for each meal for four days while the control was given sterile water absorbed into the feed. After the end of the experiment (day 4), 24 pieces of shrimp were still alive in the treated tank while 23 pieces were still alive. All shrimp were subjected to a whole-body DNA extraction.

### DNA Extraction

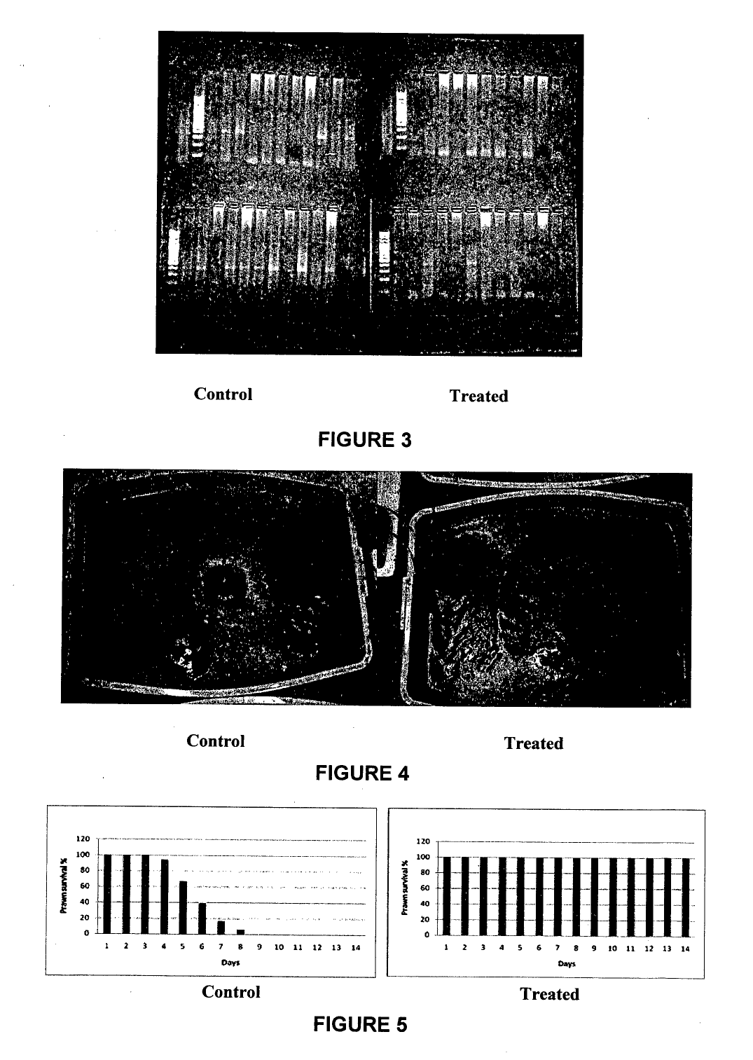

DNA was extracted from whole body using salting-procedure (Aljanabi, S. M. and L. Martinez, 1997. Universal and rapid salt-extraction of high quality genomic DNA for PCR-based techniques. Nucleic Acid Res., 25: 4692-4693). Primers used in this experiment was HPVF: 5′-ACA-CTC-AGC-CTC-TAC-CTT-GT 3′ and HPVR: 5′ -GCA-TTA-CAA-GAG-CCA-AGC-AG-3′. Thirty-five cycles of amplification were performed at 30 s at 94° C., 30 s at 55° C., and 50 s at 72° C. for both primer pairs. The expected PCR products were analyzed in a 2% agarose gel, with the expected band of 441 by as shown in FIG. 3.

### Results

PCR analysis showed that RetroMAD1 in the treated Paleonetes sp tank, 92% (22/24) were HPV negative while 8% (2/24) were HPV positive. In the control non-treated group, 95% (22/23) were HPV positive while 5 percent (1/23) were HPV negative.

### Example 3

### Effect of RetroMAD1 on WSSV-Infected Shrimp

### Shrimp Culture

White Leg Shrimp Penaeus vannamei (36 pieces) at an average of 8.0±0.5 grams were used in this experiment they were obtained from pond-reared from SPF (specific pathogen free) post-larvae obtained from commercial hatcheries. Treated sea water was obtained from the hatchery. Cultures of healthy shrimp were performed in a recirculation system (equipped with filter and aeration) with a salinity of 28-32 ppt in a bio-secure laboratory at 28° C. They were acclimatized 1 week before the infection experiment. Two groups of 18 prawns were reared in a 90 liter tank with and individual filter (FIG. 4).

### WSSV Infection

Prawns were orally challenged by feeding frozen flesh from WSSV-PCR positive prawns obtained from a recently WSSV-killed pond at approximately 5% of body weight on the first day. The next day, were given RetroMAD1 at a concentration of 0.1 mg/g body weight by coating it into a commercial feed. They were given the medicated feed for all meals (4 times a day). Observation was carried in term mortality after 24 hours of infection. At the end of the experiments, all live prawns were collected. These moribund and live prawns were subjected to PCR analysis.

### DNA Extraction

DNA was extracted from the pleopod using salting-procedure (Aljanabi, S. M. and L. Martinez, 1997). Primers used in this experiment was WSVF: 5′-TAT-TGT-CTC-TCC-TGA-CGT-AC-3′ and WSVR: 5′ -CAC-ATT-CTT-CAC-GAG-TCT-AC-3′. Thirty-five cycles of amplification were performed at 30 s at 94° C., 30 s at 55° C., and 50 s at 72° C. for both primer pairs. The expected PCR products were analyzed in a 2% agarose gel, with the expected band of 298 bp.

### Result

In the control tank, mortalities began on day 3 post-challenge and by day 8, nearly all of the 18 prawns were dead. By day 9 post-challenge, 100% mortality was observed in the control tank showing that the WSSV-infected carcass used was very much capable of causing 100% mortality within 9 days post-oral infection. In the treated tank, no mortality was observed until day fourteen.

PCR analysis showed that all moribund prawns (18/18) from the control group had high level of WSSV. Interestingly, in the treated group 9/18 had low infection, 3/18 had very low infection while 6/18 undetected (FIG. 5).

### Example 4

### Time Needed for Sero-Reversal to Occur in MBV-Infected Shrimp

Monodon Baculovirus (MBV) is an OIE ‘listed for notification’ shrimp DNA virus that has historically contributed to significant commercial losses in shrimp farming. A total of 5 MBV highly-PCR positive 6 g Penaeus vannamei were detected from a subsample of 20 shrimp obtained live from a commercial shrimp farm in Tawau, Malaysia and tested based on sacrificing one pleopod for DNA extraction. These were individually kept in separate 10 L aerated plastic aquariums that had 30% daily water exchange at 30 ppt salinity for a 1 week acclimation period. Ammonia and Nitrite were monitored to ensure adequate water quality. They were then fed a commercial pellet feed (Charoen Pokphand) once a day with 100 mg each feeding. The intentionally low feeding rate was to ensure that all the feed would be consumed and not contribute to developing ammonia in the experimental tank. RetroMAD1 at 2 mg/ml concentration was added at 150 ml/kg to prepare the stock feed for the experiment by applying it on the surface of the feed followed by convection drying at 35° C. in an oven. These were then stored at 4° C. in a refrigerator for the duration of the experiment. After a week, another pleopod was surgically removed and tested with standard Polymerase Chain Reaction (PCR) against the highly conserved coat protein of the virus. The primers used were: MBV F: 5′ TACCATAAGCTAGCATACGCC 3′ and MBV R: 5′ GGGGGCACAAGTCTCACAAG 3′. Nucleic acid isolation and the PCR protocol used were the same as Example 3 above. The size of the PCR product was 305 bp.

At the end of week 1, it was found that all the resultant pleopod samples were still PCR positive but at the end of week 2, all the samples from another surgically removed pleopod showed that they had all become PCR negative. It is therefore suggested that sero-reversal in MBV infected shrimp from PCR positive to PCR negative takes approximately 2 weeks after feeding with RetroMAD1.

TABLE 2
Results of PCR for MBV post RetroMAD1 treatment

PCR Result

| P. vannamei | Day 0 | Day 7 | Day 14 |
|---|---|---|---|
| Animal 1 | Positive | Positive | Negative |
| Animal 2 | Positive | Positive | Negative |
| Animal 3 | Positive | Positive | Negative |
| Animal 4 | Positive | Positive | Negative |

### Example 5

### Stability of RetroMAD1 to Trypsin at pH8

The ability of RetroMAD1 to withstand action of digestive enzymes acting at their pH optima is shown in Table 3 below.

50 mM DTT was prepared and added into pre-dissolved RetroMAD1 protein (1:1) made according to Example 1 and mixed. This was heated at 95° C. for 10 minutes and used to carry out enzyme assays with proteases such as Trypsin (pH8) (Lonza, Walkersville), α-Chymotrypsin (pH8) (Sigma-Aldrich) and Pepsin (pH2) (Sigma-Aldrich). After 10 minutes of heating at 95° C., the reaction was allowed to cool to room temperature (Approx. 10 mins) and proteases added to a final ratio of 1:100 (w/w) (protease:protein). This was incubated at 37° C. for 2 hours and protease activity terminated by incubating the mixture at 65° C. for 15 minutes. SDS-PAGE was used to analyze the fragments.

Other fusion proteins provided in Table 4 were made according to the method of Example 1 and the results of their fragmentation provided in Table 3.

TABLE 3
Results of fragmentation of fusion proteins according to the present invention

| | | | | | No of bands after proteas [symbol] | | |
|---|---|---|---|---|---|---|---|
| | | | | | digestion | | |
| | Size of | | SEQ ID | | | | Chymotr [symbol] |
| Drug | drug | | NO: | Structure of drug | Pepsin | Trypsin | psin |
| AM | 40 | kDa | 28 | A-B-C | No | No | No |
| | | | | (AVBD103-MAP30-MYTILINC10C) | fragment | fragment | fragment |
| CT | 36 | kDa | 29 | A-A-B-C | No | No | No |
| | | | | (CERCROPIN A-CERCROPIN D- | fragment | fragment | fragment |
| | | | | TAP29-DAP30-LATARCIN 2A) | | | |
| AB | 32 | kDa | 30 | (RETROCYCLIN 101-MORMODICA | No | No | No |
| | | | | ANTI-HIV PROTEIN 30) | fragment | fragment | fragment |
| BA | 32 | kDa | 31 | (MORMODICA ANTI-HIV PROTEIN | No | No | No |
| | | | | 30- RETROCYCLIN 101) | fragment | fragment | fragment |
| BC | 35 | kDa | 32 | (MORMODICA ANTI-HIV PROTEIN | No | No | No |
| | | | | 30- DERMASEPTIN 1) | fragment | fragment | fragment |
| CB | 35 | kDa | 33 | DERMASEPTIN 1- MORMODICA | No | No | No |
| | | | | ANTI-HIV PROTEIN 30 | fragment | fragment | fragment |
| Tamapal1 | 35.93 | kDa | 34 | C-B-C | No | No | No |
| | | | | TACHYPLESIN- MAP30- | fragment | fragment | fragment |
| | | | | ALLOFERON1 | | | |
| K5 | 36.55 | kDa | 35 | C-B-D | No | No | No |
| | | | | (GAEGURIN 5-MAP30-(KLAKLAK)2 | fragment | fragment | fragment |
| RetroMAD1 | 41.2 | kDa | 1 | A-B-C | No | No | No |
| | | | | (RETROCYCLIN 101- MAP30- | fragment | fragment | fragment |
| | | | | DERMASEPTIN 1) | | | |
| RetroGAD1 | 35.29 | kDa | 36 | A-B-C | No | No | No |
| | | | | (RETROCYCLIN 101- GAP31- | fragment | fragment | fragment |
| | | | | DERMASEPTIN 1) | | | |

[symbol]

indicates data missing or illegible when filed

TABLE 4
Examples of fusion proteins according to the present invention

| SEQ | |
|---|---|
| ID | |
| NO: | SEQUENCE |
| 27 | [G-G-G-S]ₙ |
| 28 | SFGLCRLRRGFCAHGRCRFPSIPIGRCSRFVQCCRRVWVPGVGVPGVGGATGSDVNFDLSTATAKTYTK |
| | FIEDFRATLPFSHKVYDIPLLYSTISDSRRFILLNLTSYAYETISVAIDVTNVYVVAYRTRDVSYFFKE |
| | SPPEAYNILFKGTRKITLPYTGNYENLQTAAHKIRENIDLGLPALSSAITTLFYYNAQSAPSALLVLIQ |
| | TTAEAARFKYIERHVAKYVATNFKPNLAIISLENQWSALSKQIFLAQNQGGKFRNPVDLIKPTGERFQV |
| | TNVDSDVVKGNIKLLLNSRASTADENFITTMTLLGESVVNSCASRCKGHCRARRCGYYVSVLYRGRCYC |
| | KCLRCVPGVGVPGVG |
| 29 | LEKRKWKLFKKIEKVGQRVRDAVISAGPAVATVAQATALAKNVPGVGVPGVGGATGSDVSFRLSGATSK |
| | KKVYFISNLRKALPNEKKLYDIPLVRSSSGSKATAYTLNLANPSASQYSSFLDQIRNNVRDTSLIYGGT |
| | DVAVIGAPSTTDKFLRLNFQGPRGTVSLGLRRENLYVVAYLAMDNANVNRAYYFKNQITSAELTALFPE |
| | VVVANQKQLEYGEDYQATEKNAKITTGDQSRKELGLGINLLITMIDGVNKKVRVVKDEARFLLIAIQMT |
| | AEAARFRYIQNLVTKNFPNKFDSENKVIQFQVSWSKISTAIFGDCKNGVFNKDYDFGFGKVRQAKDLQM |
| | GLLKYLGRPKSSSIEANSTDDTADVLVPGVGVPGVG KTCENLADTFRGPCFATSNC |
| 30 | MGRICRCICGRGICRCICGVPGVGVPGVGGSDVNFDLSTATAKTYTKFIEDFRATLPFSHKVYDIPLLY |
| | STISDSRRFILLDLTSYAYETISVAIDVTNVYVVAYRTRDVSYFFKESPPEAYNILFKGTRKITLPYTG |
| | NYENLQTAAHKIRENIDLGLPALSSAITTLFYYNAQSAPSALLVLIQTTAEAARFKYIERHVAKYVATN |
| | FKPNLAIISLENQWSALSKQIFLAQNQGGKFRNPVDLIKPTGERFQVTNVDSDVVKGNIKLLLNSRAST |
| | ADENFITTMTLLGESVVEFPW |
| 31 | MGSDVNFDLSTATAKTYTKFIEDFRATLPFSHKVYDIPLLYSTISDSRRFILLDLTSYAYETISVAIDV |
| | TNVYVVAYRTRDVSYFFKESPPEAYNILFKGTRKITLPYTGNYENLQTAAHKIRENIDLGLPALSSAIT |
| | TLFYYNAQSAPSALLVLIQTTAEAARFKYIERHVAKYVATNFKPNLAIISLENQWSALSKQIFLAQNQG |
| | GKFRNPVDLIKPTGERFQVTNVDSDVVKGNIKLLLNSRASTADENFITTMTLLGESVVEFPWVPGVGVP |
| | GVGGRICRCICGRGICRCICG |
| 32 | MGSDVNFDLSTATAKTYTKFIEDFRATLPFSHKVYDIPLLYSTISDSRRFILLDLTSYAYETISVAIDV |
| | TNVYVVAYRTRDVSYFFKESPPEAYNILFKGTRKITLPYTGNYENLQTAAHKIRENIDLGLPALSSAIT |
| | TLFYYNAQSAPSALLVLIQTTAEAARFKYIERHVAKYVATNFKPNLAIISLENQWSALSKQIFLAQNQG |
| | GKFRNPVDLIKPTGERFQVTNVDSDVVKGNIKLLLNSRASTADENFITTMTLLGESVVEFPWVPGVGVP |
| | GVGALWKTMLKELGTMALHAGKAALGAAADTISQGTQ* |
| 33 | MALWKTMLKELGTMALHAGKAALGAAADTISQGTQVPGVGVPGVGGSDVNFDLSTATAKTYTKFIEDFR |
| | ATLPFSHKVYDIPLLYSTISDSRRFILLDLTSYAYETISVAIDVTNVYVVAYRTRDVSYFFKESPPEAY |
| | NILFKGTRKITLPYTGNYENLQTAAHKIRENIDLGLPALSSAITTLFYYNAQSAPSALLVLIQTTAEAA |
| | RFKYIERHVAKYVATNFKPNLAIISLENQWSALSKQIFLAQNQGGKFRNPVDLIKPTGERFQVTNVDSD |
| | VVKGNIKLLLNSRASTADENFITTMTLLGESVVEFPW* |
| 34 | VPGVGVPGVGKWCFRVCYRGICYRRCRVPGVGVPGVGGATGSDVNFDLSTATAKTYTKFIEDFRATLPF |
| | SHKVYDIPLLYSTISDSRRFILLNLTSYAYETISVAIDVTNVYVVAYRTRDVSYFFKESPPEAYNILFK |
| | GTRKITLPYTGNYENLQTAAHKIRENIDLGLPALSSAITTLFYYNAQSAPSALLVLIQTTAEAARFKYI |
| | ERHVAKYVATNFKPNLAIISLENQWSALSKQIFLAQNQGGKFRNPVDLIKPTGERFQVTNVDSDVVKGN |
| | IKLLLNSRASTADENFITTMTLLGESVVNVPGVGVPGVGHGVSGHGQHGVHG |
| 35 | VPGVGVPGVGFLPLLAGLAANFLPTIICFISYKCVPGVGVPGVGGATGSDVNFDLSTATAKTYTKFIED |
| | FRATLPFSHKVYDIPLLYSTISDSRRFILLNLTSYAYETISVAIDVTNVYVVAYRTRDVSYFFKESPPE |
| | AYNILFKGTRKITLPYTGNYENLQTAAHKIRENIDLGLPALSSAITTLFYYNAQSAPSALLVLIQTTAE |
| | AARFKYIERHVAKYVATNFKPNLAIISLENQWSALSKQIFLAQNQGGKFRNPVDLIKPTGERFQVTNVD |
| | SDVVKGNIKLLLNSRASTADENFITTMTLLGESVVNVPGVGVPGVGKLAKLAK KLAKLAK |

The G.I. tract of shrimps and prawns consists of the proventriculus or gastric mill, digestive gland, midgut and its diverticula, and the rectum. Two distinct cell types occur in the digestive gland, a secretory type, and a mucopolysaccharide-containing type, whose function is not clear. The digestive gland has no intrinsic muscles, and depends on extrinsic muscles, and possibly ingested water, for filling and emptying. The midgut or hepatopancreas extends to the sixth abdominal somite and faecal material is contained in a peritrophic membrane. Defecation was at a peak 5-8 hr after food ingestion, but continued up to 20 hr. The rectum appeared to have the additional function of pumping water into the gut via the anus. As the pH of seawater is around 8, it is natural for trypsin and chymotrypsin to be the main physiological digestive enzymes whose pH optima is pH 8 unlike pepsin whose optima is at pH 2. Thus, pepsin was absent from the digestive tract of shrimps. The ability of RetroMAD1 to survive digestion by trypsin and chymotrypsin allowed it to be an effective therapeutic protein that could be administered along with the feed pellets itself thereby enabling a mode of delivery that is practical for shrimp farmers to employ. The results are shown in Table 3 above. Conjugating these peptides with MAP30, surprisingly render the fusion protein stable for oral administration as shown in its ability to survive protease digestion.

### Example 6

### Trial using Orally Delivered RetroMAD1 and an Immunostimulant Beta-Defense on Asian Seabass

Between 27 Apr. 2012 to 23 May 2012, a 4-week trial was carried out by Temanse Aquaculture S/B at its Sekayu Nursery centre in Malaysia where survivor Asian Sea Bass Lates calcarifer juveniles suffering from an unknown disease syndrome which was VNN and Iridovirus PCR-'ve were treated with individual regimes of RetroMAD1 and Beta-Defense (a commercial immune-stimulant) and a combination regime, against an untreated control. From a shipment of 30,000 fingerlings from Singapore, 20,000 quickly died within a matter of days and 240 similar sized apparently healthy animals were selected from the 10,000 survivors and were placed in 4 aquariums measuring 40×60×100cm each with 60 fingerlings for 3 days acclimation. Some minor mortalities occurred during acclimation and the experiment began with 50 fishes per batch. All water quality parameters such as Dissolved Oxygen, pH, ammonia and nitrite are regularly measured to ensure these were within normal acceptable ranges. The data is presented in Table 5 below.

TABLE 5
Results of experiment

| | Lates calcarifer survivor juveniles | | | | | | | |
|---|---|---|---|---|---|---|---|---|
| | | | | | | Weight gain and FCR | | |
| | Survival Rate % | | | | | Initial | Final | |
| Treatment | Day 1 | Day 6 | Day 14 | Day 22 | Day 28 | Weight | Weight | FCR |
| Control - feed only | 100 | 0 | 0 | 0 | 0 | 5.7 | 7.53 | n.a. |
| Treatment - feed + BD | 100 | 0 | 0 | 0 | 0 | 5.71 | 7.22 | n.a. |
| Treatment - feed + RetroMAD1 | 100 | 100 | 100 | 48 | 48 | 5.65 | 15.8 | 1.8 |
| Treatment - feed + BD + RetroMAD1 | 100 | 100 | 100 | 78 | 78 | 5.67 | 17.9 | 0.5 |

Carried out at Sekayu Nursery, Kuala Berang, Malaysia belonging to Temanse Aquaculture S/B

All control and Beta-Defense fishes died on day 6. No more mortalities occurred again until on day 22 and by day 28 at the end of the experiment, the RetroMAD1 treatment had 48% survival and the fingerlings had grown from 5.65 g-15.8 g mean weight with an FCR of 1.8. The combination treatment of RetroMAD1 gave a very convincing 78% survival with growth from 5.67-17.9 g mean weight with an FCR of 0.5. Beta-Defense was given at 45 ml per 300 g of pellet feed while RetroMAD1 was given at 0.1 ml diluted with 25 ml distilled water and added to the 300 g of feed. In the combination treatment, both were given together to 300 g of feed. Although the primary pathogen has not yet been determined, there was evidence of some secondary bacterial infection. We suspect however, that the primary pathogen is viral in nature as RetroMAD1 is a broad-spectrum antiviral oral-delivery protein drug. The efficacy shown with the 78% survival over 4 weeks also indicates that RetroMAD1 is efficacious in fishes also. Thus, there is every potential to use this method also in the production of SPF eggs and fingerlings in fish breeding.

### Example 7

### The Stability of RetroMAD1, RetroGAD1, Amatilin and Tamapal1

The polypeptides, RetroMAD1, RetroGAD1, Amatilin and Tamapal1 were capable of going through various thermocycler protocols that mimic post-extrusion processing temperatures in making extruded shrimp feed coated with RetroMAD1 and then coated again with a marine edible oil.

The fusion peptide solutions to be tested were loaded using a micropipette into 0.2 ml PCR tubes that were then placed into a thermocycler (Labnet International, MultiGene Gradient) which was then programmed to run at various temperature regimes as mentioned in Table 6. Each regime was made up of a short high temperature phase of 15 minutes followed by a longer medium temperature phase of 45 minutes. These were to mimic the actual temperature conditions when an extruded feed in the form of a wafer shaped pellet left the extrusion barrel of a twin-screw extruder which in this case is a Clextral BC45. The wafer was then sprayed with sufficient squid oil post extrusion as to form an external lipid barrier. In these thermocycler trials, the harshest condition was a 15 minute 70° C. exposure followed by a 45 minute 55° C. exposure. Samples were then run on SDS-PAGE with the lanes as follows:—Lanes: M, marker; 1, negative control treated with 2× β-mercaptoethanol positive loading dye; 2, negative control treated with 2× β-mercaptoethanol negative loading dye; 3, Sample subjected to the temperature regime and treated with 2× β-mercaptoethanol positive loading dye; 4, sample subjected to the temperature regime and treated with 2× β-mercaptoethanol negative loading dye; 5, sample subjected to the temperature regime and treated with 2× β-mercaptoethanol positive loading dye; 6, Sample subjected to the temperature regime and treated with 2× β-mercaptoethanol negative loading dye. Comparison of the gel bands against the control gave a physical evidence as to whether the protein was damaged by the heat treatment or not.

TABLE 6
Parameters of temperature fluctuations.

| Parameters | Round 1/T1 | | Round 2/T2 | | Round 3/T3 | | Round 4/T4 | |
|---|---|---|---|---|---|---|---|---|
| Temperature | 60 | 50 | 55 | 45 | 50 | 40 | 70 | 55 |
| (° C.) | | | | | | | | |
| Time (mins) | 15 | 45 | 15 | 45 | 15 | 45 | 15 | 45 |

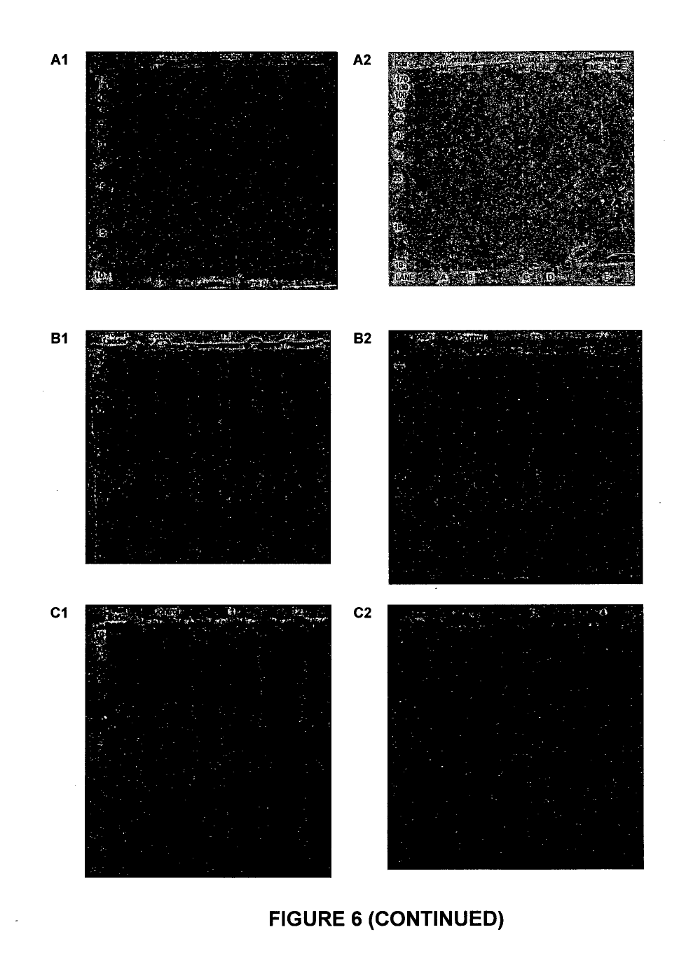

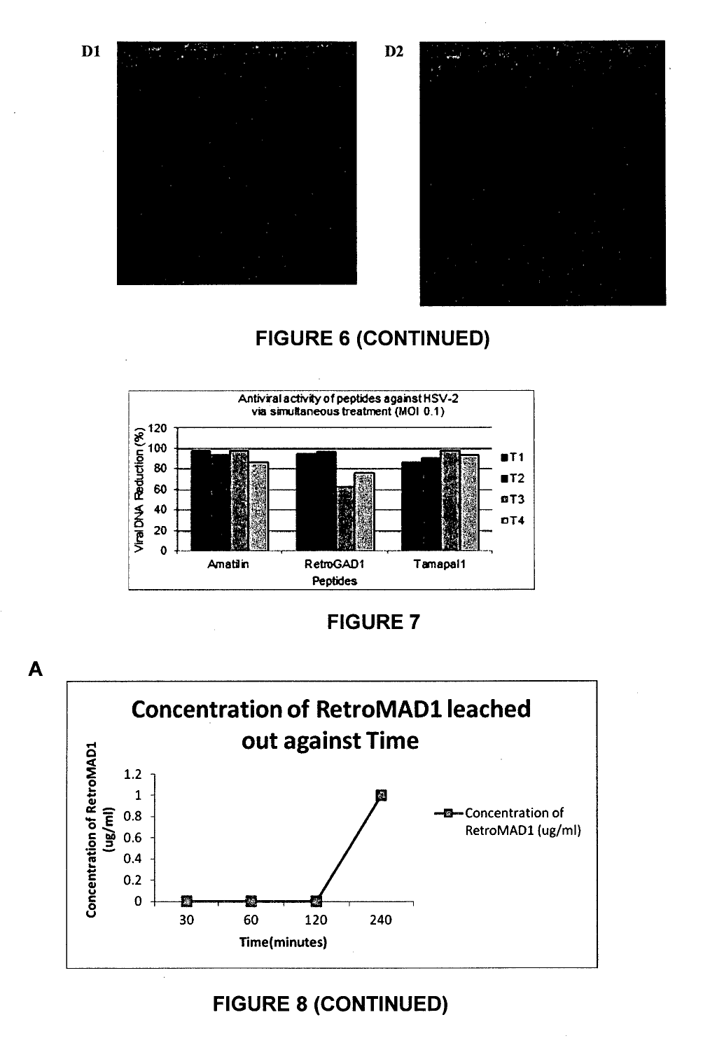

As can be seen in FIG. 6, All four drugs, RetroMAD1 (A1 and A2), RetroGAD1 (B1 and B2), Amatillin (C1 and C2) and Tamapal1 (D1 and D2) are intact under all treatments despite the presence of BME. This indicates that all four drugs are stable and will not be affected by the change in temperature.

### Example 8

### The Antiviral Activity of Peptides (Subjected to Various Temperature Fluctuations using Thermocycler) against HSV-2

Amatilin, RetroGAD1 and Tamapal1 as described in Example 7, were exposed to four sets of temperature fluctuations (T1, T2, T3 and T4) using thermocycler (Table 6 of Example 7). After exposure to various temperature fluctuations, the peptides were subjected to antiviral assay against HSV-2.

The cytotoxic activity of the peptides was quantified using MTS-based cell titer 96 non-radioactive cell proliferation assay. Briefly, monolayer cultures of Vero cells were exposed to increasing concentrations of all the three peptides for 24, 48 and 72 h of incubation. After the incubation period, the maximal concentration of the extract that did not exert toxic effect which was regarded as the maximal non-toxic concentration (MNTD) was determined using MTS assay.

After exposure to various temperature fluctuations using thermocycler (Table 6), the antiviral activity of Amatilin, RetroGAD1 and Tamapal1 was evaluated by simultaneous treatment. For simultaneous treatment the mixture of the respective peptide and virus inoculated onto Vero cells in 24-well culture plates and incubated for 24, 48 and 72 h at 37° C. under 5% CO₂ atmosphere. At the end of the time period the samples were harvested and viral DNA was extracted. The eluted DNA was then subjected to RT-PCR.

The results obtained suggest that all the three peptides were thermal stable. Amatilin and Tamapal1 showed the strongest inhibitory activity against HSV-2 at all the four set of temperature fluctuations (Table 7 and FIG. 7).

TABLE 7
Percentage of viral reduction caused by Amatilin, RetroGAD1
and Tamapal1 exposed to various temperature fluctuations
in simultaneous treatment determined by PCR.

| Set of temperature | Peptides | | |
|---|---|---|---|
| fluctuations | Amatilin | RetroGAD1 | Tamapal1 |
| T1 | 97.28 | 94.88 | 86.21 |
| T2 | 94.05 | 96.95 | 90.36 |
| T3 | 97.85 | 63.04 | 97.64 |
| T4 | 86.00 | 75.91 | 93.65 |

### Example 9

### Leaching Rate of RetroMAD1, RetroGAD1, Amatilin and Tamapal1 from the Wafer Pellets Produced in a Pilot-Scale Manufacture

The leaching rate study for as the various fusion protein drugs as described in Example 7, was to study the time points when RetroMAD1, RetroGAD1, Amatilin and Tamapal1 were leached out from the wafers. Wafers containing the drugs were placed within in 30 ppt sea salt water in 1:100 weight to volume ratio. Shrimp wafer pellets were formed by extrusion using a Clextral BC45 twin-screw extruder that was sprayed post extrusion with the fusion protein drugs to be tested followed by a spray coating in a vacuum chamber with squid oil to serve as an outer hydrophobic layer to ‘lock-in’ the test drug as well as to serve an a feeding attractant for the shrimp. Addition of RetroMAD1 was added at the rate of 300 mg/kg of wafer pellets. At 0, 30, 60, 120 and 240 minutes, sea salt water was collected to determined the concentration of the fusion protein drugs that was leached out of the wafers into the sea salt water. Capture ELISA (Promega, Glomax Multidetection System) was used to determine the concentration of RetroMAD1, while Direct ELISA was used for RetroGAD1, Amatilin and Tamapal1. In Capture ELISA, a 96 U-bottom well plated was coated with 1:1000 of rabbit anti-RetroMAD1 antibody and was incubated at 4° C. overnight. The plate was then washed with PBS-Tween20 six times before adding the samples collected at time point 0, 30, 60, 120 and 240 minutes and incubated at 37° C. for an hour. Subsequently, 1:2500 human anti-RetroMAD1 antibodies were added to capture RetroMAD1 from the samples bound on the rabbit anti-RetroMAD1 antibody. While in direct ELISA, a 96 well U-bottomed plate was coated with the samples collected at time point 0, 30, 60, 120 and 240 minutes and incubated overnight at 4° C. The plate was then washed with PBS-Tween20 and added with 1:500 rabbit antibodies against RetroGAD1, Amatilin and Tamapal1 to capture the protein drug bound on the plate. Subsequently, 1:10000 anti-rabbit IgG were added to detect rabbit antibodies bind against the protein drugs. Absorbance was read at 490 nm and 600 nm. A standard curve of drug concentration against absorbance was plotted to determine the concentration of the drug in each sample.

Both RetroMAD1 and Tamapal1 began leaching out only after 120 minutes. Both Amatilin and RetroGAD1 did not show any signs of leaching even 240 minutes. This shows that since shrimp usually consume all their feed within 30-60 minutes, this method of oral administration of these fusion protein drugs is viable for the treatment of shrimp viruses. Furthermore, as shrimp digestion is trypsin rather than chymotrypsin dependent, it does not matter that the drug is presented along with the feed.

Antibodies toward RetroMAD1, RetroGAD1, Amatilin and Tamapal1 were raised in 4 rabbits respectively. In each immunization, rabbits were immunized with RetroMAD1, RetroGAD1, Amatilin and Tamapal1 in single dose of 0.6 ml per rabbit which is a dose of 0.2 mg/kg body weight for RetroMAD1, 0.9 ml per rabbit which is a dose of 0.25 mg/kg body weight for RetroGAD1, 0.8 ml per rabbit which is a dose of 0.25 mg/kg body weight for Amatilin and 1 ml per rabbit which is a dose of 0.25 mg/kg body weight for Tamapal1.

Prior to immunization, on Day 0, blood was drawn from the rabbits. Pre-bleed blood collected on Day 0 was used as the base line in determining the antibody titer in rabbit. After pre-bleeding the rabbit, first immunization was given according to the dosage per body weight as mentioned above. Rabbits were bled before giving another immunization on Day7, Day 14, Day 28 and Day 35. Blood serum of rabbits collected on Day7, Day 14, Day 28 and Day 35 was used to determine antibody titer against RetroMAD1, RetroGAD1, Amatilin and Tamapall. On Day 38, antibody towards RetroMAD1, RetroGAD1, Amatilin and Tamapal1 raised in rabbits were harvested. In harvesting the rabbit antibody, before bleeding, each rabbit was given anesthesia (Ketamine and Xylazine) intravenously; the sedative dose was calculated using the following formula

Ketamine=(30×body weight of the rabbit)/(Concentration of Ketamine, 100 mg/ml)

Xylazine=(3×body weight of the rabbit)/(Concentration of Xylazine, 20 mg/ml)

50 ml of blood was collected from each rabbit. Blood was centrifuged at 4000 rpm for 15 minutes; blood serum containing antibody towards RetroMAD1, RetroGAD1, Amatilin and Tamapal1 was collected and kept in −20° C. for further use.

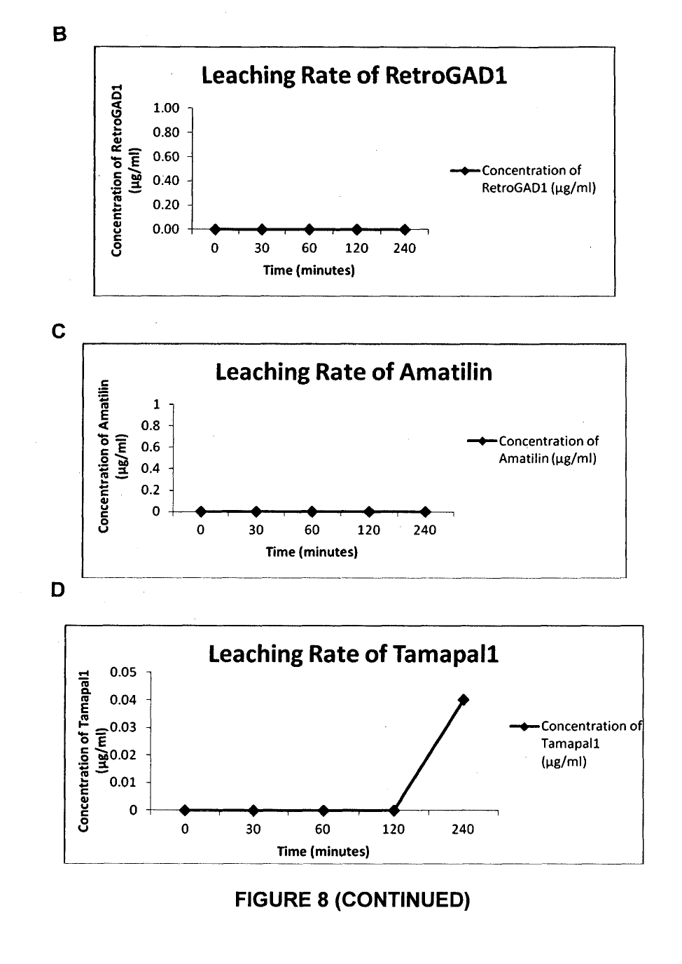

A direct ELISA was used to determine antibody titer in rabbit serum. A 96-well U-bottomed plate was coated with 1 μg/ml of RetroMAD1, RetroGAD1, Amatilin and Tamapal1 in coating buffer (0.2 M sodium carbonate-bicarbonate, ph 9.6). The sample coated plate was incubated at 4° C. overnight. Plates were washed six times with 0.05% Tween-20 in PBS 1×. 100 ∥l of 1/10 rabbit serum was added to the well, a ½ serial dilution of the rabbit serum was made. Rabbit serum was diluted in 1/10, 1/20, 1/40, 1/80, 1/160, 1/320, 1/640, 1/1280, 1/2560, 1/5120 and 1/10240 to determine the antibody titer. After incubation at 37° C. for 1 hour, plates were washed similarly and 100 μl/well of anti-rabbit IgG diluted 1:10000 in 5% BSA in PBS was added. After incubation at 37° C. for 1 hour, plates were washed and 100 μl/well streptavidin-HRP diluted 1:20000 in 5% BSA in PBS was added. After incubation at 37° C. for 1 hour in the dark, plates were washed and 100 μl/well of OPD added to each well. Plates were incubated in the dark for 30 min at room temperature and reaction stopped with 50 μl/well of 4N H₂SO₄. Optical densities (OD) were measured at 490 nm and 600 nm as background. The results are shown in FIG. 8A-D and tables 8-11.

TABLE 8
Concentration of RetroMAD1 leached out against time

| Time (minutes) | Concentration of RetroMAD1 (μg/ml) |
|---|---|
| 30 | 0 |
| 60 | 0 |
| 120 | 0 |
| 240 | 1 |

TABLE 9
Concentration of RetroGAD1 leached out at 0, 30, 60, 120 and 240
minutes

| | Concentration of RetroGAD1 |
|---|---|
| Time (Minutes) | (μg/ml) |
| 0 | 0.00000 |
| 30 | 0.00000 |
| 60 | 0.00000 |
| 120 | 0.00000 |
| 240 | 0.00000 |
| Control | 0.00000 |

TABLE 10
Concentration of Amatilin leached out at 0, 30, 60, 120 and 240 minutes

| Time (Minutes) | Concentration of Amatilin (μg/ml) |
|---|---|
| 0 | 0.00000 |
| 30 | 0.00000 |
| 60 | 0.00000 |
| 120 | 0.00000 |
| 240 | 0.00000 |
| Control | 0.00000 |

TABLE 11
Concentration of Tamapal1 leached out at 0, 30, 60, 120 and 240 minutes

| Time (Minutes) | Concentration of Tamapal1 (μ/ml) |
|---|---|
| 0 | 0.000000 |
| 30 | 0.000000 |
| 60 | 0.000000 |
| 120 | 0.000000 |
| 240 | 0.040167 |
| Control | 0.000000 |

### Example 10

### Short-Term Pharmacokinetics of RetroMAD1 in Shrimp using Capture ELISA

In the short-term feeding study, shrimps were fed with 0.06 g of shrimp wafer pellets containing RetroMAD1 at an inclusion of 300 mg/kg. This study is to determine the short term kinetics of RetroMAD1 in terms of absorption, retention and excretion. Shrimp wafer pellets were formed by extrusion using a Clextral BC45 twin-screw extruder that was sprayed post extrusion with the fusion protein drugs to be tested followed by a spray coating in a vacuum chamber with squid oil to serve as an outer hydrophobic layer to ‘lock-in’ the test drug as well as to serve an a feeding attractant for the shrimp. Addition of RetroMAD1 was added at the rate of 300 mg/kg of wafer pellets.

Healthy specimens of the commonly cultured Pacific white shrimp Penaeus vannamei were selected from a shrimp farm in Tawau, Sabah, Malaysia and a single specimen ranging from 2.4-5.8 g was placed in each transparent plastic aquarium tank of 10 litres total capacity containing 5 litres of seawater at 32 parts per thousand salinity. Specimens were acclimated for a week prior to the experiment and 50% water was changed daily by siphoning. A single airstone was provided such that aeration was sufficiently provided such that the animal did not display any signs of being stressed. A plastic netting was provided on top to prevent the specimens from jumping out. For each sampling time point, tanks were present in triplicate as in Group 1, 2 and 3. As there were 8 sampling time points, 24 tanks were prepared as shown in the Table 12.

TABLE 12
Experiment design for measuring short-term pharmacokinetics of
RetroMAD1 in shrimp

| Sampling Points | Number of Shrimp per tank | | |
|---|---|---|---|
| Time (Hours) | Group 1 | Group 2 | Group 3 |
| Control | 1 | 1 | 1 |
| 0.5 | 1 | 1 | 1 |
| 1 | 1 | 1 | 1 |
| 1.5 | 1 | 1 | 1 |
| 2 | 1 | 1 | 1 |
| 3 | 1 | 1 | 1 |
| 5 | 1 | 1 | 1 |
| 8 | 1 | 1 | 1 |

At each sampling time point, the feces were collected by siphoning, the shrimp dissected removing the hepatopancreas well as the muscle of the last abdominal segment of the tail which was stored in PBS buffer and stored at −40° C. Note that the Control were fed normal shrimp pellets without RetroMAD1. The shrimp were unfed for the duration of the experiment after completely ingesting the test and control feeds. The weights of the feces, hepatopancreas and tail muscle (only the last abdominal segment) collected are presented in the table 13 below.

TABLE 13
Weight of each shrimp, hepatopancreas, tail muscle (last segment only)
and feces Captured ELISA (Promega, Glomax Multidetection System)
was used to determine concentration of RetroMAD1 in the samples.
The tail muscle sampled was in the last abdominal segment after the
anus to ensure any result did not come from the GI tract. In captured
ELISA, a 96 U-bottom well plated was coated with 1:1000 of rabbit anti-
RetroMAD1 antibody (as mentioned in Example 9) and was incubated
at 4° C. overnight. Plate was then washed with PBS-Tween20 six
times before adding the samples of hepatopancreas, tail muscle
and feces and incubated at 37° C. for an hour. Subsequently,
1:2500 human anti-RetroMAD1 antibody was added to capture
RetroMAD1 from the samples bound on the rabbit anti-RetroMAD1
antibody. Absorbance was read at 490 nm and 600 nm. A standard
curve of concentration of RetroMAD1 (μg/ml) against absorbance
as shown in Table 13 was plotted to determine the concentration of
RetroMAD1 in each sample.

| | | Weight (grams) | | | |
|---|---|---|---|---|---|
| Time | | | | Tail | |
| (Hour) | Tank | Whole Shrimp | Hepatopancreas | Muscle | Feces |
| 0 (Control) | 1 | 3.238 | 0.120 | 0.108 | 0.000 |
| | 2 | 3.227 | 0.160 | 0.127 | 0.038 |
| | 3 | 3.554 | 0.190 | 0.231 | 0.043 |
| 0.5 | 1 | 3.600 | 0.250 | 1.960 | 0.045 |
| | 2 | 2.440 | 0.170 | 0.142 | 0.040 |
| | 3 | 3.720 | 0.187 | 0.136 | 0.020 |
| 1 | 1 | 2.785 | 0.152 | 0.100 | 0.065 |
| | 2 | 3.213 | 0.141 | 0.133 | 0.030 |
| | 3 | 4.236 | 0.211 | 0.208 | 0.015 |
| 1.5 | 1 | 2.130 | 0.126 | 0.095 | 0.070 |
| | 2 | 4.117 | 0.175 | 0.206 | 0.053 |
| | 3 | 1.612 | 0.100 | 0.083 | 0.086 |
| 2 | 1 | 5.500 | 0.236 | 0.222 | 0.041 |
| | 2 | 2.784 | 0.155 | 0.116 | 0.052 |
| | 3 | 2.993 | 0.182 | 0.160 | 0.056 |
| 3 | 1 | 3.538 | 0.190 | 0.142 | 0.024 |
| | 2 | 3.719 | 0.175 | 0.154 | 0.083 |
| | 3 | 3.995 | 0.199 | 0.147 | 0.054 |
| 5 | 1 | 3.508 | 0.154 | 0.188 | 0.060 |
| | 2 | 3.962 | 0.194 | 0.200 | 0.016 |
| | 3 | 2.443 | 0.155 | 0.104 | 0.050 |
| 8 | 1 | 4.995 | 0.245 | 0.245 | 0.035 |
| | 2 | 5.840 | 0.182 | 0.292 | 0.034 |
| | 3 | 3.460 | 0.148 | 0.146 | 0.038 |

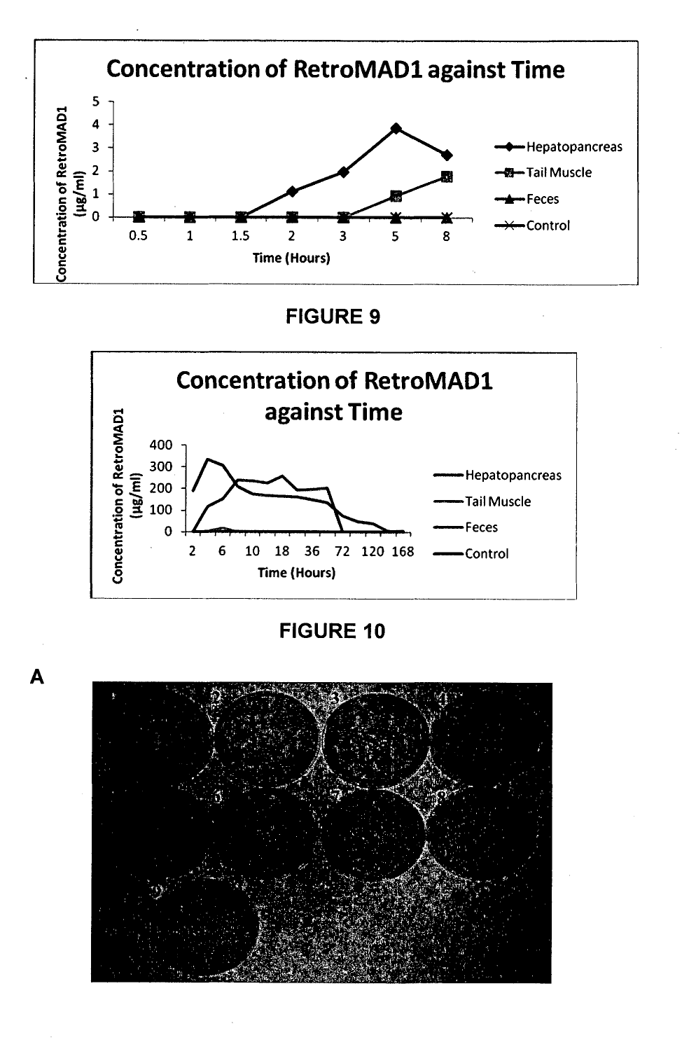

Table 14 and FIG. 9 show that hepatopancreal absorption of RetroMAD1 was detectable at 1.5 hours post-feeding and peaked at 5 hours post-feeding while RetroMAD1 was detectable in the tail muscle as early as 3 hours post-feeding.

TABLE 14
Concentration of RetroMAD1 against time

| | Treated with RetroMAD1 | | | |
|---|---|---|---|---|
| Time (hours) | Hepatopancreas | Tail muscle | Feces | Control |
| 0.5 | 0 | 0 | 0 | 0 |
| 1 | 0 | 0 | 0 | 0 |
| 1.5 | 0 | 0 | 0 | 0 |
| 2 | 1.133 | 0 | 0 | 0 |
| 3 | 1.983 | 0 | 0 | 0 |
| 5 | 3.867 | 0.9333 | 0 | 0 |
| 8 | 2.717 | 1.783 | 0 | 0 |

### Example 11

### Long-Term Pharmacokinetics of RetroMAD1 in Shrimp using Capture ELISA

In the long-term feeding study, shrimps were fed with 0.2 g of shrimp pellets containing RetroMAD1 at 300 mg/kg inclusion rate. This study is to further determine the pharmacokinetics of RetroMAD1 in terms of absorption, retention and excretion over 7 days.

In this study, Healthy specimens of the commonly cultured Pacific white shrimp Penaeus vannamei were selected from a shrimp farm in Tawau, Sabah, Malaysia and 8 pcs of 10.0 g +/−0.5 g specimen were placed in a single transparent plastic aquarium tank of 50 litres total capacity containing 40 litres of seawater at 32 parts per thousand salinity. Specimens were acclimated for a week prior to the experiment and 50% water was changed every alternate day by siphoning. A single airstone was provided such that aeration was sufficiently provided such that the animal did not display any signs of being stressed. A plastic netting was provided on top to prevent the specimens from jumping out. Feces were collected daily by siphoning and placed into a 1.5 ml plastic tube that was capped and stored at −40° C. until required. For each sampling time point, tanks were present in triplicate as in Group 1,2 and 3. As there were 7 sampling time points as well as one control, 24 tanks were prepared each with 8 specimens as shown in Table 15:

TABLE 15
Experiment design for measuring long-term pharmacokinetics of
RetroMAD1 in shrimp

| Sampling Points | Number of Shrimps per tank | | |
|---|---|---|---|
| Time (Days) | Group 1 | Group 2 | Group 3 |
| Control | Control 1 | Control 2 | Control 3 |
| 1 | 8 | 8 | 8 |
| 2 | 8 | 8 | 8 |
| 3 | 8 | 8 | 8 |
| 4 | 8 | 8 | 8 |
| 5 | 8 | 8 | 8 |
| 6 | 8 | 8 | 8 |
| 7 | 8 | 8 | 8 |

Within the first day, there were 6 sampling points at 2, 4, 6, 8, 12 and 16 hours. As there were 8 animals, one was removed at each sampling point and the feces and dissected hepatopancreas as well as the tail muscle from the last abdominal segment pooled and kept in PBS at −40° C. till required. The sampling point at 24 hours was taken at the start of Day 2 while the 36 hours sampling point taken mid-way through Day 2. Thereafter, there were sampling points daily. Captured ELISA (Promega, Glomex Multidetection System) was used to determine concentration of RetroMAD1 in hepatopancreas, tail muscle and faeces, in the method previously described earlier.

Table 16 and FIG. 10 showed that hepatopancreal RetroMAD1 peaked after 5 hours in agreement with the previous experiment (Example 10). Residual RetroMAD1 could be detected up to 144 hours (day 6) even after faecal RetroMAD1 was no long detectable after 72 hours (day 3). Detectable RetroMAD1 in the tail muscle remained only for 8 hours showing that the drug residue is quickly broken down by active swimming shrimp.

This indicated a safe ‘withdrawal period’ of 14 days for head-on shrimp product (sold with the head containing the hepatopancreas) and 7 days for a headless shrimp product (where the head containing the hepatopancreas is removed at the processing factory prior to freezing.

TABLE 16
Concentration of RetroMAD1 in hepatopancreas, tail muscle and
feces at 2, 4, 6, 8, 10, 12, 18, 24, 36, 48, 72, 96, 120, 144 and
168 hours.

| | Concentration of RetroMAD1 (μg/ml) | | | |
|---|---|---|---|---|
| Time (Hours) | Hepatopancreas | Tail Muscle | Feces | Control |
| 2 | 189.62 | 0 | 0 | 0 |
| 4 | 335.085 | 5.272 | 115.751 | 0 |
| 6 | 304.758 | 16.015 | 151.794 | 0 |
| 8 | 209.824 | 0 | 238.178 | 0 |
| 10 | 174.754 | 0 | 234.733 | 0 |
| 12 | 168.082 | 0 | 224.924 | 0 |
| 18 | 165.378 | 0 | 256.926 | 0 |
| 24 | 160.538 | 0 | 194.13 | 0 |
| 36 | 147.025 | 0 | 195.96 | 0 |
| 48 | 136.772 | 0 | 201.383 | 0 |
| 72 | 75.827 | 0 | 0 | 0 |
| 96 | 49.567 | 0 | 0 | 0 |
| 120 | 40.343 | 0 | 0 | 0 |
| 144 | 3.275 | 0 | 0 | 0 |
| 168 | 3.426 | 0 | 0 | 0 |

### Example 12

### An Orally Administered WSSV Challenge to Post-Larvae Treated with Various Fusion Proteins

Protein concentrations were determined by Bradford Analysis (Quick Start Bradford Protein Assay, http://www.bio-rad.com/webroot/web/pdf/lsr/literature/4110065A.pdf). The post-larvae were acclimatized for 1 week, continued with pre-infection period of 3 weeks and post-infection period of 1 week. The post-larvae were fed throughout the duration. Feeding was done 3 times a day mixed with the drugs at a ratio of 150 ml/kg feed. RetroMAD1 and Tamapal1 were effective against the WSSV oral infection as shown in Tables 16-19. No mortality was observed until Day 30.

Feed was observed to be completely consumed justifying the increase in feeding rate over the period of the experiment. Tamapal1 differs from Tamapal1 (A) by use of a different refolding buffer.

Tail muscle tissue (20 mg) was used to determine the viral number retained in the shrimp. DNA extraction was carried out using a ‘salting-out’ method (Miller, S. A.; Dykes, D. D.; Polesky, H. F. (1988). “A simple salting out procedure for extracting DNA from human nucleated cells”. Nucleic acids research 16 (3): 1215). Tissues were lysed in 600 μl of lysis buffer (25 mM EDTA, 2% SDS) containing 1.5 μl of 20 mg/ml proteinase K. The lysate was incubated at 65° C. for 45 minutes or until the solid tissue have been completely dissolved. The lysate was treated with 1.5 μl of 4 mg/ml RNase A for another 15 minutes followed by cooling to room temperature for 5 minutes. Protein precipitation solution (200 μl) was added and the lysate was vortexed for 25 seconds followed by incubation on ice for 5 minutes. The homogenate was centrifuged at 13,000×g got 10 minutes and 600 μl of supernatant was transferred to a 1.5 ml centrifuge containing 600 μl of isopropanol. The tube was inverted gently 40 times. DNA was precipitated at 13,000×g for 5 minutes, the supernatant was discarded. The DNA was further washed by adding 600 μl of 70% ethanol and centrifuged for another 3 minutes. Finally the supernatant was discarded and the tube was left to air-dry for 15 minutes. TE buffer was added at 100 μl and used for PCR.

In this study, three sense primers (F1 [SEQ ID NO:40]: 5′ ATGGATTTGGCAACCTAACA 3′, F2 [SEQ ID NO:41]: 5′ AATTCGTGGAGAGAGGTCC 3′ and F3 [SEQ ID NO:42]: 5′ ATCTCTACCGTCACACAGCC 3′) and 1 antisense primer (R1 [SEQ ID NO:43]: 5′ GAAGATTTTAATGTCCTTGCTCG 3′) were designed from the nucleotide sequence of a 626 by WSSV viral product (van Hulten M. C. W., Witteveldt J., Peters S., Kloosterboer N., Tarchini R., Fiers M., Sand brink H., Klein Lankhorst R. and Vlak J. M. 2001. The white spot syndrome virus DNA genome sequence. Virol 67: 233-241).

The F1 and R1 primers were used as external primers to generate a primary PCR product of 500 base pairs (bp) while F2 and F3 were used internal primers along with R1 to generate nested PCR product of 300 and 200 bp, respectively. The 30 μl PCR reaction contained 50 mM KCI, 10 mM Tris-HCl, pH 9.0, 1.5 mM MgCl, 0.2 mM each of deoxy (d) ATP, dCTP, dGTP and dTTP, 1 μM of RI, 0.3 μM of F1, 0.3 μM of F2 and 0.4 μM of F3, 1.25 U Taq polymerase. The PCR reaction initiated by heating the mixture 95° C. for 5 min followed by 30 cycles of 30 s at 95° C., 30 s at 55° C. and 30 s at 72° C. with a final extension of 10 min at 72° C. Following PCR, the amplified products were analyzed by electrophoresis in 2% agarose gel stained with ethidium bromide and visualized by ultraviolet transillumination.

TABLE 16
Post larvae mortality rate.

| | Mortality (times) | | | | | | |
|---|---|---|---|---|---|---|---|
| | 24 | 48 | 72 | 96 | 120 | 144 | 168 |
| Tanks | hours | hours | hours | hours | hours | hours | hours |
| Control (+) 1 | 0 | 6 | 9 | 10 | 10 | 10 | 10 |
| Control (+) 2 | 0 | 2 | 10 | 10 | 10 | 10 | 10 |
| Tamapal1 1 | 0 | 2 | 8 | 9 | 9 | 9 | 9 |
| Tamapal1 2 | 0 | 0 | 0 | 0 | 0 | 0 | 0 |
| Tamapal1 (A) 1 | 0 | 3 | 9 | 9 | 9 | 9 | 9 |
| Tamapal1 (A) 2 | 0 | 5 | 8 | 10 | 10 | 10 | 10 |
| RetroGAD1- 1 | 0 | 2 | 10 | 10 | 10 | 10 | 10 |
| RetroGAD1- 2 | 0 | 1 | 8 | 10 | 10 | 10 | 10 |
| Amatilin 1 | 0 | 4 | 10 | 10 | 10 | 10 | 10 |
| Amatilin 2 | 0 | 4 | 10 | 10 | 10 | 10 | 10 |
| RetroMAD1-1 | 0 | 0 | 0 | 0 | 0 | 0 | 0 |
| RetroMAD1-2 | 0 | 0 | 0 | 0 | 0 | 0 | 0 |
| Control (−) 1 | 0 | 0 | 0 | 0 | 0 | 0 | 0 |
| Control (−) 2 | 0 | 0 | 0 | 0 | 0 | 0 | 0 |

TABLE 17
PCR result at the end of trial period. (Each
set of drugs were tested in duplicates.)

| | Pcs | | | | | | | | | |
|---|---|---|---|---|---|---|---|---|---|---|
| Tanks | 1 | 2 | 3 | 4 | 5 | 6 | 7 | 8 | 9 | 10 |
| Control (+) 1 | H | H | H | H | M | H | H | H | H | H |
| Control (+) 2 | H | H | H | H | H | H | H | H | H | H |
| Tamapal1 1 | H | H | H | H | H | H | H | H | H | |
| Tamapal1 2 | — | — | — | — | — | — | — | — | — | — |
| Tamapal1 (A) 1 | H | H | M | H | H | M | H | H | H | H |
| Tamapal1 (A) 2 | H | H | H | H | H | M | L | L | H | H |
| RetroGAD1-1 | H | H | M | H | H | H | H | H | H | H |
| RetroGAD1-2 | H | H | H | H | H | H | H | H | H | H |
| Amatilin 1 | H | H | H | M | H | H | H | H | H | H |
| Amatilin 2 | H | H | H | M | H | H | M | H | M | H |
| RetroMAD1-1 | — | — | — | — | — | — | — | — | — | — |
| RetroMAD1-2 | — | — | — | — | — | — | — | — | — | — |
| Control (−) 1 | — | — | — | — | — | — | — | — | — | — |
| Control (−) 2 | — | — | — | — | — | — | — | — | — | — |

H: High; M: Medium; L: Low; and —: no band observed.

TABLE 18
Concentration of drugs used.

| | Concentration |
|---|---|
| | (mg/ml) |
| Tamapal1 | 0.468 |
| Tamapal1 (A) | 1.43 |
| RetroGAD1 | 0.58 |
| Amatilin | 0.615 |
| RetroMAD1 | 1.14 |

TABLE 19
Feeding rate from Day 1-30.

| | | Amount of drug in μg ingested by a test animal | | | | |
|---|---|---|---|---|---|---|
| Pre & post | Feed/day/ | assuming even feeding ** | | | | |
| infection | aquarium* | Tamapal1 | Tamapal1 (A) | RetroGAD1 | Amatilin | RetroMAD1 |
| Day 1-4 | 0.09 g | 0.63 | 1.9305 | 0.783 | 0.83025 | 1.539 |
| Day 5-9 | 0.12 g | 0.8424 | 2.574 | 1.044 | 1.107 | 2.052 |
| Day 10-16 | 0.18 g | 1.2636 | 3.861 | 1.566 | 1.6605 | 3.078 |
| Day 17-30 | 0.27 g | 1.8954 | 5.7915 | 2.349 | 2.4907 | 4.617 |

*10 pieces of post larvae per aquarium
** Even feeding assumes that all test animals ingested at the same rate.

### Example 13

### Amatilin having Antibacterial Activity on Vibrio cholera and Vibrio parahaemolyticus from In Vitro Assay

The minimum inhibitory concentration (MIC) of Amatilin against Vibrio cholera and Vibrio parahemolyticus was performed according to the Clinical and Laboratory Standard Institute guidelines using the broth micro dilution method. Stock Amatilin at concentration of 1290 μg/ml was two-fold serially diluted in cationically adjusted Mueller Hinton broth (CAMHB) in 96 well round bottom plate to 50 μl. Bacterial cultures from −80° C. glycerol stock were passaged twice on nutrient agar and resuspended in phosphate buffered saline (PBS) to OD₆₂₅ 0.08-0.1 which was equivalent to 1-2×10⁸ cfu/ml. The suspension was adjusted to 1×10⁶ cfu/ml and added in equal volume (50 μl) to the 96 well plate prepared with the serial dilutions of Amatilin. Final testing concentration of Amatilin ranged from 2.52 μg/ml to 322.5 μg/ml. Plates were incubated for 18-24 hours under 37° C. MIC was read as the lowest concentration of drug that completely inhibits the visible growth of the bacteria. Following that, aliquot of 10 μl from each well was serially ten-fold diluted in PBS and plated on Mueller Hinton agar for viable colony count. Wells with bacterial density of more than 1×10¹⁰ cfu/ml was noted as “>1×10¹⁰ cfu/ml”, implying no

| | | Cell count | | | |
|---|---|---|---|---|---|
| | | | 24 hr | | |
| | Concentration | | | | Actual cell |
| Bacteria | (microgram/ml) | Time (hr) | Cell counted^{a} | Dilution factor | no. (cfu/ml)^{b} |
| Vibrio cholerae | Untreated | 0 | 188 | 10³ | 1.88 × 10⁵ |
| | Untreated | 24 | Overcrowded | 10¹⁰ | >1 × 10¹⁰ |
| | 322.5 | 24 | 0 | 10² | 0 |
| | 161.25 | 24 | 51 | 10² | 5.1 × 10³ |
| | 80.63 | 24 | 124 | 10⁹ | >1 × 10¹⁰ |
| | 40.31 | 24 | Overcrowded | | |
| | 20.16 | 24 | Overcrowded | | |
| | 10.08 | 24 | Overcrowded | | |
| | 5.04 | 24 | Overcrowded | | |
| | 2.52 | 24 | Overcrowded | | |
| Vibrio | Untreated | 0 | 26 | 10⁴ | 2.6 × 10⁵ |
| parahaemolyticus | Untreated | 24 | Overcrowded | 10¹⁰ | >1 × 10¹⁰ |
| | 322.5 | | 0 | 1 | 0 |
| | 161.25 | | 0 | 1 | 0 |
| | 80.63 | | 27 | 10⁸ | 2.7 × 10⁹ |
| | 40.31 | | Overcrowded | 10¹⁰ | >1 × 10¹⁰ |
| | 20.16 | | Overcrowded | 10¹⁰ | >1 × 10¹⁰ |
| | 10.08 | | Overcrowded | 10¹⁰ | >1 × 10¹⁰ |
| | 5.04 | | Overcrowded | 10¹⁰ | >1 × 10¹⁰ |
| | 2.52 | | Overcrowded | 10¹⁰ | >1 × 10¹⁰ |

^{a}Plates with more than 200 colonies were denoted as “overcrowded”.
^{b}Actual cell no. (cfu/ml) more than 1 × 10¹⁰ were denoted as “>1 × 10¹⁰”.

inhibitory activity. Results as shown in FIGS. 11A and B and tables 20 and 21 were pooled from duplicate tests in three independent trials.

TABLE 20
MIC of Amatilin

| Bacteria | MIC (μg/ml) |
|---|---|
| Vibrio cholerae | 161.25 (10.08) |
| Vibrio parahaemolyticus | 161.25 (161.25) |

Table 21: Antibacterial Effect of Amatilin on V. cholerae and V. parahemolyticus.

### Example 14

### Antiviral Activity of Amatilin, RetroGAD1, RetroMAD1 and Tamapal1 using HSV2

### Cytotoxicity of Tested Peptides on Vero Cells

The effect of Amatilin, RetroGAD1, RetroMAD1 and Tamapal1 on the growth of Vero cells was examined to rule out any direct cytotoxicity. Monolayer cultures of Vero cells were exposed to increasing concentrations of Amatilin, RetroGAD1, RetroMAD1 and Tamapall. After 24, 48 and 72 h of incubation, cell viability was determined using MTS assay. Results shown indicate the accepted maximal nontoxic concentrations of the four peptides on Vero cells. At the chosen

Maximal non-toxic dose (MNTD) as shown in Table 22, the peptides did not impair the cell viability with respect to the untreated control group.

TABLE 22
Maximal non-toxic dose of the peptides on Vero cells

| | MNTD (μg/ml) | | |
|---|---|---|---|
| Peptide | 24 h | 48 h | 72 h |
| Amatilin | 15 | 15 | 15 |
| RetroGAD1 | 10 | 10 | 10 |
| RetroMAD1 | 50 | 50 | 50 |
| Tamapal1 | 15 | 15 | 15 |

The Antiviral Activity of Peptides against HSV-2

The antiviral activity of Amatilin, RetroGAD1, RetroMAD1 and Tamapal1 was evaluated by simultaneous treatment. For simultaneous treatment the mixture of the respective peptide and virus was inoculated onto Vero cells and incubated for 24, 48 and 72 h at 37° C. under 5% CO₂ atmosphere. At the end of the time period the samples were harvested and viral DNA was extracted. The eluted DNA was then subjected to RT-PCR.

The results obtained suggested that all the four peptides have strong inhibitory activity against HSV-2 via simultaneous treatment at the maximal non-toxic dose (MNTD) (Table 22). Amatilin showed 99.88, 99.99 and 99.98% of inhibition, respectively, after 24, 48 and 72 h. RetroGAD1 exhibited 95.45, 91.71 and 89.95% inhibitory activity, respectively, at 24, 48 and 72 h.

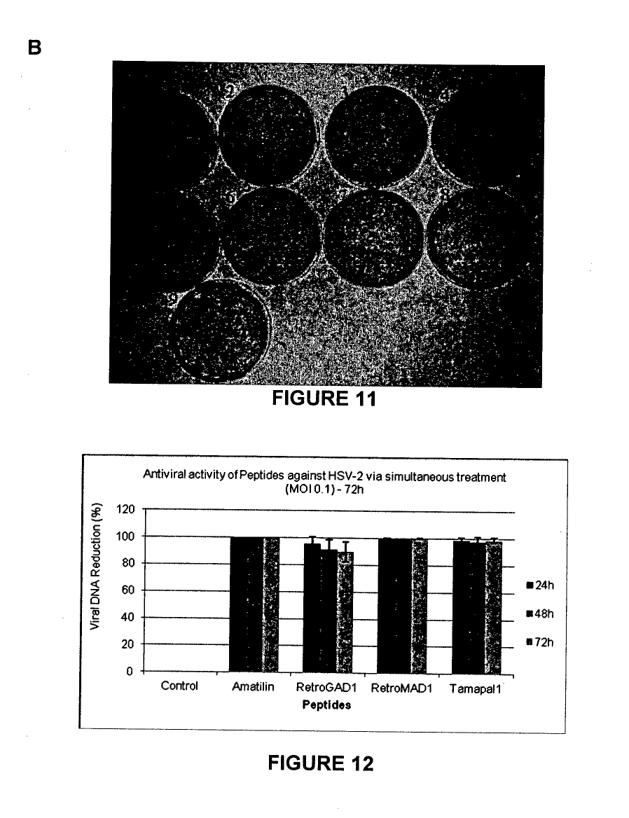

RetroMAD1 showed 99.67, 99.96 and 99.87% of viral reduction, respectively, at 24, 48 and 72 h. Tamapal1 showed 98.75, 98.00 and 98.98% inhibition, respectively, at 24, 48 and 72 h (Table 23 and FIG. 12).

TABLE 23
Percentage of viral reduction caused by Amatilin, RetroGAD1,
RetroMAD1 and Tamapal1 in simultaneous treatment at 72 h
determined by PCR.

| | Time | | |
|---|---|---|---|
| Peptides | 24 h | 48 h | 72 h |
| Amatilin | 99.88 | 99.99 | 99.98 |
| RetroGAD1 | 95.45 | 91.71 | 89.95 |
| RetroMAD1 | 99.67 | 99.96 | 99.87 |
| Tamapal1 | 98.75 | 98.00 | 98.98 |

### Example 15

### Effects of Temperature on the Stability of Amatilin, RetroGAD1 and Tamapal1 Evaluated Via Antiviral Activity

Amatilin, RetroGAD1 and Tamapal1 were placed in −20, 4, 26 and 37° C. for up to 30 days. The peptides were also placed in 50° C. for up to 7 days. To further investigate the thermostability of the peptides, they were also exposed to four set of various temperature fluctuations. Subsequently, the peptides were analyzed for their antiviral activity against HSV-2 via simultaneous treatment.

The antiviral activity of peptides (subjected to various temperatures) against HSV-2 The antiviral activity of Amatilin, RetroGAD1 and Tamapal1 after incubation at different temperatures (−20, 4, 26, 37 and 50° C.) for 1, 7 and 30 days was evaluated by simultaneous treatment. For simultaneous treatment the mixture of the respective peptide and virus inoculated onto Vero cells and incubated for 24, 48 and 72h at 37° C. under 5% CO₂ atmosphere. At the end of the time period the samples were harvested and viral DNA was extracted. The eluted DNA was then subjected to RT-PCR.

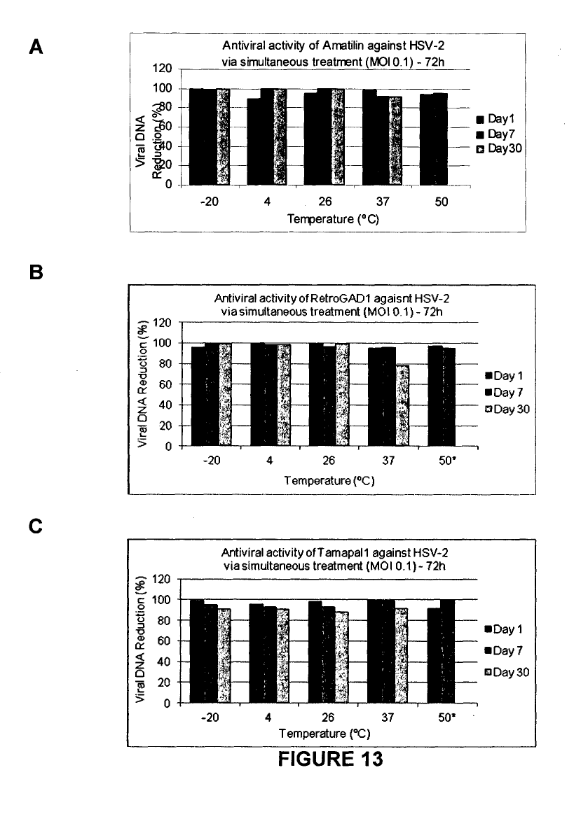

The results obtained suggest that all the three peptides were thermal stable. The peptides exposed to various temperatures for 1, 7 and 30 days showed strong inhibitory activity against HSV-2 via simultaneous treatment at the maximal non-toxic dose (MNTD) (Table 20 in Example 14). Amatilin was stable at high temperatures, 26 and 37° C. for up to 30 days giving 99.95 and 91.78% of inhibition, respectively. Amatilin was also stable at 50° C. for up to 7 days with 94.75% inhibitory activity (Table 24 and FIG. 13A). RetroGAD1 exhibited 99.01 and 78.52% inhibitory activity, respectively, after incubation at 26 and 37° C. for up to 30 days. The peptide showed 95.03% of viral reduction after incubation at 50° C. for up to 7 days (Table 24 and FIG. 13B). Tamapal1 was stable for up to 30 days at 26 and 37° C. giving 88.12 and 91.78% inhibitory activity, respectively. The peptide remained stable for 7 days at 50° C. with 99.42% of viral reduction (Table 24 and FIG. 13C).

TABLE 24
Percentage of viral reduction caused by Amatilin, RetroGAD1 and Tamapal1 incubated at different
temperatures for 1, 7 and 30 days in simultaneous treatment determined by PCR.

| | Peptides | | | | | | | | |
|---|---|---|---|---|---|---|---|---|---|
| Temperature | Amatilin | | | RetroGAD1 | | | Tamapal1 | | |
| (° C.) | Day 1 | Day 7 | Day 30 | Day 1 | Day 7 | Day 30 | Day 1 | Day 7 | Day 30 |
| −20 | 99.84 | 98.00 | 99.98 | 95.93 | 98.94 | 98.96 | 98.73 | 94.77 | 91.01 |
| 4 | 89.35 | 99.98 | 99.92 | 99.66 | 98.92 | 98.30 | 95.84 | 92.92 | 90.87 |
| 26 | 94.53 | 99.75 | 99.95 | 99.77 | 96.24 | 99.01 | 98.49 | 92.92 | 88.12 |
| 37 | 98.55 | 91.45 | 91.78 | 95.54 | 95.61 | 78.52 | 99.45 | 99.16 | 91.78 |
| 50 | 94.31 | 94.95 | — | 97.12 | 95.03 | — | 91.36 | 99.42 | — |

### Example 16

### Method of Micronizing RetroMAD1 into a Free-Flowing Powder by Supercritical Fluid Drying (SCFD)

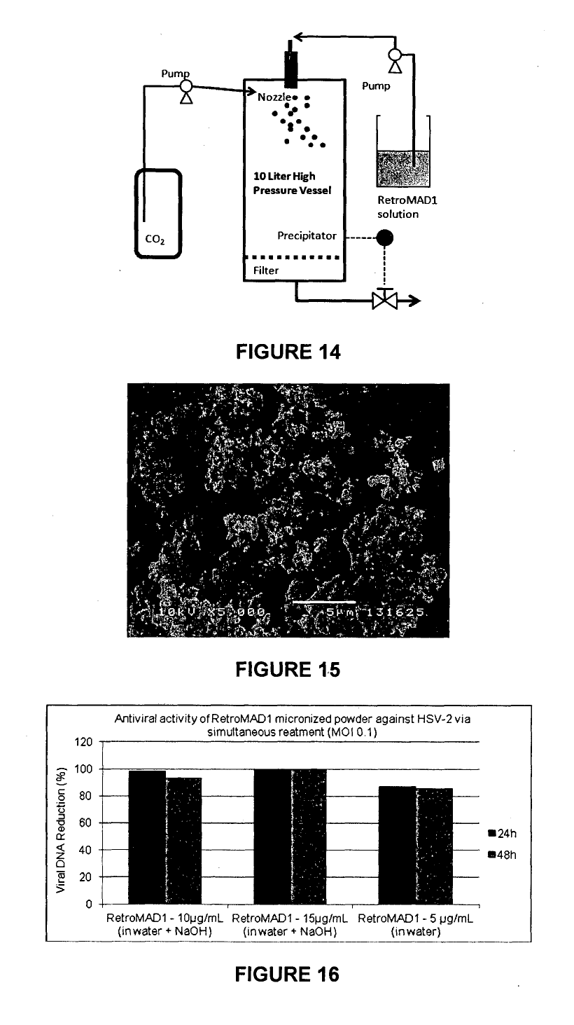

A 10 L high pressure vessel of the configuration conventionally used for Supercritical Fluid Drying (SCFD) was used to dry and produce a micronized form of powdered free-flowing RetroMAD1 under the conditions of 120 bar; 37 C; 300 kg/hr CO2 flow that gave 88-89% yield in 2 cases and a lower 58% yield in one case due to operator error. The resulting powder was observed to be slightly cubic when viewed under Scanning Electron Microscope (SEM) and about 1 micron in size on the average. This confirms that RetroMAD1 may be efficiently manufactured as a powder for incorporation into tablets, capsules and animal feed pellets whether for terrestrial or aquatic application. The schematics of the process is shown in FIG. 14 and an SEM picture showing the morphology of the RetroMAD1 crystals is shown in FIG. 15.

Bioactivity of RetroMAD1 SCFD Micronized Powder Evaluated using HSV-2

The bioactivity of the micronized form of powdered free-flowing RetroMAD1 produced using

SCFD was tested via antiviral assay against HSV-2. For the antiviral test, RetroMAD1 micronized powder was dissolved in two different solvents: (i) ultra pure water with 5.5 mM NaOH; and (ii) ultra pure water. Ultra pure water was produced using a Sastec ST-WP-UVF machine.

### Cytotoxicity of RetroMAD1 Micronized Powder

Prior to screening RetroMAD1 micronized powder for its antiviral properties, it was subjected to cytotoxicity assay in order to identify the maximal concentration, which could be non-toxic to Vero cells. The cytotoxic activity of the peptides was quantified using MTS-based cell titer 96 non-radioactive cell proliferation assay. Briefly, monolayer cultures of Vero cells were exposed to increasing concentrations of the dissolved RetroMAD1 powder for 24, 48 and 72h of incubation. After the incubation period, the maximal concentration of the protein that did not exert toxic effect is regarded as the maximal non toxic concentration (MNTD) was determined using MTS assay.

Results as shown in Table 25 indicate that the accepted maximal nontoxic concentrations of RetroMAD1 micronized powder on Vero cells were less than 20 μg/ml. At the chosen MNTD, the peptides did not impair the cell viability with respect to the untreated control group.

TABLE 25
Maximal non-toxic dose of RetroMAD1 micronized powder on Vero cells

| | MNTD, ug/ml | | |
|---|---|---|---|
| Peptide | 24 h | 48 h | 72 h |
| RetroMAD1 powder (in water + NaOH) | 15 | 15 | 15 |
| RetroMAD1 powder (in water) | 5 | 5 | 5 |

The Antiviral Activity of RetroMAD1 Micronized Powder against HSV-2

The antiviral activity of RetroMAD1 micronized powder was evaluated by simultaneous treatment. For simultaneous treatment, the mixture of RetroMAD1 and virus were inoculated onto Vero cells in 24-well culture plates and incubated for 24 and 48h at 37° C. under 5% CO₂ atmosphere. At the end of the time period the samples were harvested and viral DNA was extracted. The eluted DNA was then subjected to RT-PCR.

The results obtained suggested that RetroMAD1 in powder form exhibited strong inhibitory activity against HSV-2 via simultaneous treatment giving between 85% -100% of inhibition. RetroMAD1 powder was dissolved in ultrapure water with NaOH showed higher percentage of viral reduction compared to the powder dissolved in ultroapure water alone at the MNTD (Table 26 and FIG. 16). SCFD was therefore a viable method of producing RetroMAD1 in a solid dose format good for incorporation into tablets, capsules, medicated chewing gum and aquatic feed pellets.

TABLE 26
Percentage of viral reduction caused by RetroMAD1 micronized powder in
simultaneous determined by PCR.

Time

| RetroMAD1 | 24 h | 48 h |
|---|---|---|
| RetroMAD1 (in water + NaOH) - 10 μg/ml | 98.33 | 93.06 |
| RetroMAD1 (in water + NaOH) - 15 μg/ml | 100.00 | 100.00 |
| RetroMAD1 (in water) - 5 μg/ml | 87.13 | 85.43 |

### REFERENCES

1. Hizer S. E. et. al. (2002). RAPD markers as predictors of infectious hypodermal and haematopoietic necrosis virus (IHHNV) resistance in shrimp (Litopenaeus stylirostris). Genome 45(1): 1-7;
2. Liu H. et. al. (2009). Antiviral immunity in crustaceans. Fish Shellfish Immunol. 2009 27(2):79-88.
3. Argue B. et. al. (2002). Selective breeding of Pacific white shrimp (Litopenaeus vannamei) for growth and resistance to Taura Syndrome Virus. Aquaculture 204(3-4): 447-460.
4. Aljanabi, S. M. and L. Martinez, 1997. Universal and rapid salt-extraction of high quality genomic DNA for PCR-based techniques. Nucleic Acid Res., 25: 4692-4693;
Sambrook and Green, Molecular Cloning: A Laboratory Manual, Cold Springs Harbor Laboratory (Fourth Edition), New York (2012),
5. Tang Y Q, Yuan J, Osapay G et al. (October 1999). “A cyclic antimicrobial peptide produced in primate leukocytes by the ligation of two truncated alpha-defensins”. Science 286 (5439): 498-502;
6. Leonova L, Kokryakov V N, Aleshina G et al. (September 2001). “Circular minidefensins and posttranslational generation of molecular diversity”. J. Leukoc. Biol. 70 (3): 461-4.
7. Wang W et al Activity of alpha- and theta-defensins against primary isolates of HIV-1. Journal of Immunology 173(1): 515-520 (2004),
8. U.S. Pat. No. 8,076,284 B2;
9. Kim, S. et al, Peptides, 2003, 24, 945-953.
10. Fan, J-M., et al, Mol Biotechnol, 2008, 39, 79-86.
11. Karlin & Altschul (1993) Proc. Natl. Acad. Sci. USA 90: 5873-5877
12. Brudno M., Bioinformatics 2003b, 19 Suppl 1:154-162.

1. A method of producing at least one specific pathogen free (SPF) non-human animal, the method comprising:
(a) selecting a surviving animal in an environment comprising at least one pathogen that is capable of infecting and/or killing the animal;
(b) administration of a fusion protein to the surviving animal wherein the fusion protein comprises at least one polypeptide B which is a Type 1 Ribosome Inactivating Protein (RIP) or fragment thereof; and
(i) at least one polypeptide A which is an Antimicrobial peptide; and/or
(ii) at least one polypeptide C which is a Cationic AntiMicrobial Peptide (CAP) or fragment thereof; and
(c) resulting surviving animal is the SPF non-human animal.

2. The method according to claim 1, further comprising a step of confirming that the surviving animal from step (a) expresses at least one marker of a pathogen resistant gene before the administration of the fusion protein of step (b).

3. The method according to claim 1, wherein the surviving animal is at least one animal selectively bred for growth.

4. The method according to claim 1, wherein the presence of the SPF non-human animal in step (c) is confirmed using at least one of the methods selected from the group consisting of PCR, ELISA, LAMP and RT-PCR.

5. The method according claim 1, wherein the non-human animal is at least one aquatic animal.

6. The method according to claim 5, wherein the aquatic animal is at least one crustacean.

7. (canceled)

8. The method according to claim 2, wherein the marker is selected from the group consisting of pmAV, c-type lectin, haemocyanin, beta-integrin, syntenin, alpha-2-macroglobulin, LPS-binding protein, beta-glucan binding protein, catalase gene, Ras-related nuclear protein, caspace-3 like gene, calreticulin, Rab GTPase gene, and Mg-SOD gene.

9. The method according to claim 1, wherein the administration is by oral delivery.

10. The method according to claim 1, wherein the fusion protein is administered with food.

11. The method according to claim 1, wherein the SPF non-human animal is free from at least one pathogen selected from the group consisting of Avian influenza viruses, Lymphoid Leukosis, Visceral Leukosis (Marek's Disease), Quail Bronchitis viruses, Newcastle disease viruses, infectious bronchitis viruses, infectious Bursal disease viruses, rhinoviruses, echoviruses, equine encephalitis viruses, coronaviruses, vesicular stomatitis viruses, rabies viruses, ebola viruses, parainfluenza viruses, Hanta viruses, bunga viruses, phleboviruses and Nairo viruses, hemorrhagic fever viruses, reoviruses, orbiviurses and rotaviruses, parvoviruses, papilloma viruses, polyoma viruses, adenoviruses, Aquabirnaviruses, Betanoda viruses, Salmonid alphaviruses, Epizotic Hematopoietic necrosis viruses, Infectious salmon anemia viruses (ISAV), Nervous necrosis viruses, Abalone Viral ganglioneuritis, Abalone Herpes-like viruses, variola viruses, vaccinia viruses, pox viruses, African swine fever virus, Iridovirus, Infectious Salmonid Anaemia (ISA), White Spot Syndrome Virus (WSSV), Hepatopancreactic parvo-like virus (HPV), Monodon Baculo virus (MBV), Infectious Hypodermal and Hematopoietic Necrosis Virus (IHHNV), Yellow Head Virus (YHV), Taura syndrome virus (TSV), Gill-associated virus (GAV), Laem-Singh Virus (LSNV), Infectious Myonecrosis Virus (IMNV), Mourilyan virus (MoV), Koi herpesvirus 1 (KHV 1), KHV2, KHV3, viral nervous necrosis (VNN), infectious pancreatic necrosis virus (IPNV), channel catfish virus (CCV), fish lymphocystis disease virus (FLDV), infectious hematopoietic necrosis virus (IHNV) and viral hemorrhagic septicemia virus (VHSV), AMAV, swine hepatitis E virus, Circoviruses, Herpesviruses, Porcine cytomegalovirus, pseudorabies virus, Feline Panleukopenia virus (FPV), Feline herpesvirus, Feline calicivirus, Feline Leukemia Virus (FeLV), Feline Immunodeficiency Virus (FIV), Rabies virus, canine parvovirus, canine coronavirus, canine distemper virus, canine influenza, canine hepatitis virus, canine herpesvirus, a virus that causes pseudorabies, canine minute virus and a bacteriophage.

12. The method according to claim 11, wherein the bacteriophage is selected from a group consisting of Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttavirus, Inoviridae, Leviviridae, Microviridae, Plasmaviridae, Tectiviridae and the like. In particular, the phage may be Lambda phage (λphage)-lysogen (λphage), T2 phage, T4 phage, T7 phage, T12 phage, R17 phage, M13 phage, MS2 phage, G4 phage, P1 phage, Enterobacteria phage P2, P4 phage, Phi X 174 phage, N4 phage, Pseudomonas phage Φ6, Φ29 phage, 186 phage and the like.

13. The method according to claim 1, wherein the polypeptide A is a defensin and selected from the group consisting of alpha, beta, theta, a member of the big defensins protein family, an analogue, and a fragment thereof.

14. The method according to claim 1, wherein the fusion protein comprises the structure A-B-C, A-C-B, C-A-B, C-B-A, B-A-C, B-C-A, A-B-C-C, A-B, B-A, B-C, C-B, C-B-C, or C-C-B-C-C.

15. The method according to claim 1, wherein the fusion protein comprises polypeptides A, B and C.

16. The method according to claim 1, further comprising at least one linker peptide between each of the polypeptides A, B and/or C.

17. The method according to claim 16, wherein the linker peptide has SEQ ID NO: 3 or 35.

18. The method according to claim 1, wherein polypeptide A is:
(a) a theta defensin selected from the group consisting of Rhesus minidefensin (RTD-1), RTD-2, RTD-3, Retrocyclin-1, Retrocyclin-2, Retrocyclin-3, synthetic retrocyclin congener RC100, RC101, RC102, RC103, RC104, RC105, RC106, RC107, RC108, RC110, RC111, RC112, RC113 and RC114; or
(b) an alpha-defensin selected from the group consisting of human neutrophil protein 1 (HNP-1), HNP-2, HNP-3, HNP-4, Human defensin 5 and Human defensin 6, an analogue, or a fragment thereof; or
(c) a beta-defensin selected from the group consisting of DEFB 1, DEFB 4A, DEFB 4B, DEFB 103A, DEFB 103B, DEFB 104A, DEFB 104B, DEFB 105A, DEFB 105B, DEFB 106A, DEFB 106B, DEFB 107A, DEFB 107B, DEFB 108B, DEFB108 P1-4, DEFB 109 P1, DEFB 109 P1B, DEFB 109 P2-3, DEFB 110, DEFB 112-119 and DEFB 121-136.

19. (canceled)

20. (canceled)

21. The method according to claim 1, wherein the Type 1 RIP (polypeptide B) is selected from the group consisting of Ebulitins, Nigritins, Amarandins, Amaranthus antiviral/RIP, Amaranthin, Atriplex patens RIP, Beta vulgaris RIP, β-vulgin, Celosia cristata RIP, Chenopodium album RIP, CAP30B, Spinacea oleracea RIP, Quinqueginsin, Asparins, Agrostin, Dianthins, DAPs, Dianthus chinensis', Lychnin, Petroglaucin, Petrograndin, Saponaria ocymoides RIP, Vacuolas saporin, Saporins, Vaccaria hispanica RIP, Benincasins, Hispin, Byrodins, Colocins, Cucumis figarei RIP, Melonin, C. moschata RIP, Cucurmo sin, Moschatins, Pepocin, Gynostemmin, Gynostemma pentaphyllum RIP, Gypsophilin, Lagenin, Luffaculin, Luffangulin, Luffin, MORs, Momordin II, Momorcharins, Momorcochin, Momorcochin-S, Sechiumin, Momorgrosvin, Trichoanguin, Kirilowin, α-trichosanthin, TAP-29, Trichokirin, Trichomislin, Trichosanthin, Karasurin, Trichomaglin, Trichobakin, Crotin, Euserratin Antiviral Protein GAP-31, Gelonin, Hura crepitans RIP, Curcin, Jathropa curcas RIP, Mapalmin, Manutins, α-pisavin, Charibdin, Hyacinthus orientalis RIP, Musarmin, Iris hollandica RIP, Cleroendrum aculeatum RIP, CIPs),Crip-31, Bouganin, Bougainvilla spectbilis RIP, Bougainvillea×buttiana Antiviral protein 1 (BBAP1), Malic enzymes, MAP-S, pokeweed antiviral proteins (PAP), PD-SI, DP-S2, Dodecandrin, PIP, PIP2, Phytolacca octandra anti-viral proteins, Hordeum vulgare RIP's, Hordeum vulgare sub sp. Vulgare Translational inhibitor II, Secale cereale RIP, Tritin, Zea diploperemis RIPs, Malus×domestica RIP, Momordica Anti-HIV Protein, Gelonium multiflorum, Mirabilis expansa 1, phage MU1, betavulgin (Bvg), curcin 2, saporin 6, Maize RIP (B-32), Tobacco RIP (TRIP), Beetins, Mirabilis antiviral protein (MAP), Trichosanthin (TCS), luffins, Momorcharins, Ocymoidin, Bryodin, Pepopsin, β-trichosanthin, Camphorin, YLP, Insularin, Barley RIP, Tritins, Lamjarin, and Volvariella volvacea RIP.

22. The method according to claim 1, wherein the CAP (polypeptide C) is selected from the group consisting of Cyclotides, Siamycins, NP-06, Gramicidin A, Circulins, Kalatas, Ginkbilobin, Alpha-Basrubin, Lunatusin, Sesquin, Tricyclon A, Cycloviolacins, Polyphemusins, hfl-B5, Protegrins (Pig Cathelicidin), Rat Defensins, Human β-defensins, Temporins, Caerins, Ranatuerins, Reptile Defensin, Piscidins, Lactoferricin B, Rabbit Neutrophils, Rabbit α-Defensin, Retrocyclins, Human α-Defensins, Human β-defensin III (HBD3), Rhesus minidefensin (RTD-1,θ-defensin), rhesus θ-defensins, Human neutrophil peptides, Cecropin As, Melittin, EP5-1, Magainin 2s, hybrid (CE-MA), hepcidin TH1-5, Epinecidin-1, Indolicidin, Cathelicidin-4, LL-37 Cathelicidin, Dermaseptins, Maximins, Brevinins, Ranatuerins, Esculentins, Maculatin 1.3, Maximin H5 and Piscidins, Mundticin KS Enterocin CRL-35, Lunatusin, FK-13 (GI-20 is a derivative), Tachyplesins, Alpha-MSH, Antiviral protein Y3, Palustrin-3AR, Ponericin L2, Spinigerin, Melectin, Clavanin B, Cow cathelicidins, Guinea pig cathelicidin CAP11, Sakacin 5X, Plectasin, Fungal Defensin, GLK-19, lactoferrin (Lf) peptide 2, Alloferon 1, Uperin 3.6, Dahlein 5.6, Ascaphin-8, Human Histatin 5, Guineapig neutrophils, Mytilins, EP5-1, Hexapeptide (synthetic) Corticostatin IV Rabbit Neutrophil 2, Aureins, Latarcin, Plectasin, Cycloviolins, Vary Peptide E, Palicourein, VHL-1, and Buforins.

23. The method according to claim 1, wherein:
(a) the Type 1 RIP is MAP30, the CAP is Dermaseptin 1 and the polypeptide A is Retrocyclin 101; or
(b) the Type 1 RIP is MAP30, the CAP is Alloferonl and the polypeptide A is Tachyplesin; or
(c) the Type 1 RIP is MAP30, the CAP is Mytillin C10C and the polypeptide A is AVBD103; or
(d) the Type 1 RIP is GAP31, the CAP Dermaseptin1 and the polypeptide A Retrocyclin 101.

24. The method according to claim 23, wherein the fusion protein in (a) comprises the amino acid sequence SEQ ID NO: 1; in (b) comprises the amino acid sequence SEQ ID NO: 34; in (c) comprises the amino acid sequence SEQ ID NO: 28; and in (d) comprises the amino acid sequence SEQ ID NO: 36.

25-30. (canceled)

31. A method of producing at least one specific pathogen resistant (SPR) non-human animal, the method comprising:
(a) producing a specific pathogen free animal according to any one of claims 1-30,
(b) selective breeding of a male and female SPF non-human animal to produce a SPR non-human animal offspring.

32. A specific pathogen free non-human animal according to claim 1.

33. A specific pathogen resistant non-human animal according to claim 31.


SEQUENCE LISTING (43 sequences)

SEQ ID NO: 1 - protein, 379 aa, Artificial Sequence
  Polypeptide sequence of Amatilin
MKYLLPTAAAGLLLLAAQPAMAMGRICRCICGRGICRCICGVPGVGVPGVGGATGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPWALWKTMLKELGTMALHAGKAALGAAADTISQGTQVPGVGVPGVGKLAAALEHHHHHH

SEQ ID NO: 2 - DNA, 1140 bp, Artificial Sequence
  Coding sequence of Amatilin
atgaaatacctgctgccgaccgctgctgctggtctgctgctcctcgctgcccagccggcgatggccatggggcgtatttgccgttgcatttgcggccgtggcatttgccgctgcatctgtggcgtgccgggtgttggtgttccgggtgtgggtggtgcgaccggatccgatgtgaactttgatctgagcaccgcgaccgcgaaaacctataccaaattcatcgaagattttcgtgcgaccctgccgtttagccataaagtgtatgatatcccgctgctgtatagcaccattagcgatagccgtcgttttattctgctggatctgaccagctatgcgtatgaaaccattagcgtggcgattgatgtgaccaacgtgtatgtggtggcgtatcgtacccgtgatgtgagctactttttcaaagaaagcccgccggaagcgtacaacattctgtttaaaggcacccgtaaaattaccctgccgtataccggcaactatgaaaacctgcagaccgcggcgcataaaattcgtgaaaacatcgatctgggcctgccggccctgagcagcgcgattaccaccctgttttattataacgcgcagagcgcgccgagcgcgctgctggtgctgattcagaccaccgcggaagcggcgcgttttaaatatattgaacgccacgtggcgaaatatgtggcgaccaactttaaaccgaacctggccattattagcctggaaaaccagtggagcgccctgagcaaacaaatttttctggcccagaaccagggcggcaaatttcgtaatccggtggatctgattaaaccgaccggcgaacgttttcaggtgaccaatgtggatagcgatgtggtgaaaggcaacattaaactgctgctgaacagccgtgcgagcaccgcggatgaaaactttattaccaccatgaccctgctgggcgaaagcgtggtggaattcccgtgggcgctgtggaaaaccatgctgaaagaactgggcaccatggcgctgcatgcgggtaaagcggcgctgggtgcggcagcggataccattagccagggcacccaggttccgggcgtgggcgttccgggcgttggtaagcttgcggccgcactcgagcaccaccaccaccaccactga

SEQ ID NO: 3 - protein, 5 aa, Artificial Sequence
  A polypeptide of the present invention
VPXVG

SEQ ID NO: 4 - protein, 18 aa, Artificial Sequence
  A polypeptide of the present invention
GRICRCICGRGICRCICG

SEQ ID NO: 5 - protein, 268 aa, Artificial Sequence
  A polypeptide of the present invention
GSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPW

SEQ ID NO: 6 - protein, 34 aa, Artificial Sequence
  A polypeptide of the present invention
ALWKTMLKELGTMALHAGKAALGAAADTISQGTQ

SEQ ID NO: 7 - protein, 18 aa, Artificial Sequence
  A polypeptide sequence of naturally occurring and synthetic theta Defensin proteins
GFCRCLCRRGVCRCICTR

SEQ ID NO: 8 - protein, 18 aa, Artificial Sequence
  A polypeptide sequence of naturally occurring and synthetic theta Defensin proteins
RCLCRRGVCRCLCRRGVC

SEQ ID NO: 9 - protein, 18 aa, Artificial Sequence
  A polypeptide sequence of naturally occurring and synthetic theta Defensin proteins
RCICTRGFCRCICTRGFC

SEQ ID NO: 10 - protein, 18 aa, Artificial Sequence
  A polypeptide sequence of naturally occurring and synthetic theta Defensin proteins
GICRCICGRGICRCICGR

SEQ ID NO: 11 - protein, 18 aa, Artificial Sequence
  A polypeptide sequence of naturally occurring and synthetic theta Defensin proteins
GICRCICGRGICRCICGR

SEQ ID NO: 12 - protein, 18 aa, Artificial Sequence
  A polypeptide sequence of naturally occurring and synthetic theta Defensin proteins
RICRCICGRRICRCICGR

SEQ ID NO: 13 - protein, 18 aa, Artificial Sequence
  A polypeptide sequence of naturally occurring and synthetic theta Defensin proteins
GICRCICGRGICRCICGR

SEQ ID NO: 14 - protein, 18 aa, Artificial Sequence
  A polypeptide sequence of naturally occurring and synthetic theta Defensin proteins
GICRCICGKGICRCICGR

SEQ ID NO: 15 - protein, 18 aa, Artificial Sequence
  A polypeptide sequence of naturally occurring and synthetic theta Defensin proteins
GICRCYCGRGICRCICGR

SEQ ID NO: 16 - protein, 18 aa, Artificial Sequence
  A polypeptide sequence of naturally occurring and synthetic theta Defensin proteins
GICRCICGRGICRCYCGR

SEQ ID NO: 17 - protein, 18 aa, Artificial Sequence
  A polypeptide sequence of naturally occurring and synthetic theta Defensin proteins
GYCRCICGRGICRCICGR

SEQ ID NO: 18 - protein, 18 aa, Artificial Sequence
  A polypeptide sequence of naturally occurring and synthetic theta Defensin proteins
GICRCICGRGYCRCICGR

SEQ ID NO: 19 - protein, 18 aa, Artificial Sequence
  A polypeptide sequence of naturally occurring and synthetic theta Defensin proteins
GICYCICGRGICRCICGR

SEQ ID NO: 20 - protein, 18 aa, Artificial Sequence
  A polypeptide sequence of naturally occurring and synthetic theta Defensin proteins
GICICICGYGICRCICGR

SEQ ID NO: 21 - protein, 18 aa, Artificial Sequence
  A polypeptide sequence of naturally occurring and synthetic theta Defensin proteins
GICICICGRGICYCICGR

SEQ ID NO: 22 - protein, 18 aa, Artificial Sequence
  A polypeptide sequence of naturally occurring and synthetic theta Defensin proteins
GICICICGRGICYCICGR

SEQ ID NO: 23 - protein, 18 aa, Artificial Sequence
  A polypeptide sequence of naturally occurring and synthetic theta Defensin proteins
RGCICRCIGRGCICRCIG

SEQ ID NO: 24 - protein, 18 aa, Artificial Sequence
  A polypeptide sequence of naturally occurring and synthetic theta Defensin proteins
RGCICRCIGRGCICRCIG

SEQ ID NO: 25 - protein, 18 aa, Artificial Sequence
  A polypeptide sequence of naturally occurring and synthetic theta Defensin proteins
GICRCICGRGICRCICGR

SEQ ID NO: 26 - protein, 18 aa, Artificial Sequence
  A polypeptide sequence of naturally occurring and synthetic theta Defensin proteins
GICRCICGKGICRCYCGR

SEQ ID NO: 27 - protein, 4 aa, Artificial Sequence
  An example of fusion proteins according to the present invention
GGGS

SEQ ID NO: 28 - protein, 360 aa, Artificial Sequence
  An example of fusion proteins according to the present invention
SFGLCRLRRGFCAHGRCRFPSIPIGRCSRFVQCCRRVWVPGVGVPGVGGATGSDVNFDLSTATAKTYTKFIEDFRATLPFSHKVYDIPLLYSTISDSRRFILLNLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVNSCASRCKGHCRARRCGYYVSVLYRGRCYCKCLRCVPGVGVPGVG

SEQ ID NO: 29 - protein, 401 aa, Artificial Sequence
  An example of fusion proteins according to the present invention
LEKRKWKLFKKIEKVGQRVRDAVISAGPAVATVAQATALAKNVPGVGVPGVGGATGSDVSFRLSGATSKKKVYFISNLRKALPNEKKLYDIPLVRSSSGSKATAYTLNLANPSASQYSSFLDQIRNNVRDTSLIYGGTDVAVIGAPSTTDKFLRLNFQGPRGTVSLGLRRENLYVVAYLAMDNANVNRAYYFKNQITSAELTALFPEVVVANQKQLEYGEDYQAIEKNAKITTGDQSRKELGLGINLLITMIDGVNKKVRVVKDEARFLLIAIQMTAEAARFRYIQNLVTKNFPNKFDSENKVIQFQVSWSKISTAIFGDCKNGVFNKDYDFGFGKVRQAKDLQMGLLKYLGRPKSSSIEANSTDDTADVLVPGVGVPGVGKTCENLADTFRGPCFATSNC

SEQ ID NO: 30 - protein, 297 aa, Artificial Sequence
  An example of fusion proteins according to the present invention
MGRICRCICGRGICRCICGVPGVGVPGVGGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPW

SEQ ID NO: 31 - protein, 297 aa, Artificial Sequence
  An example of fusion proteins according to the present invention
MGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPWVPGVGVPGVGGRICRCICGRGICRCICG

SEQ ID NO: 32 - protein, 313 aa, Artificial Sequence
  An example of fusion proteins according to the present invention
MGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPWVPGVGVPGVGALWKTMLKELGTMALHAGKAALGAAADTISQGTQ

SEQ ID NO: 33 - protein, 313 aa, Artificial Sequence
  An example of fusion proteins according to the present invention
MALWKTMLKELGTMALHAGKAALGAAADTISQGTQVPGVGVPGVGGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPW

SEQ ID NO: 34 - protein, 328 aa, Artificial Sequence
  An example of fusion proteins according to the present invention
VPGVGVPGVGKWCFRVCYRGICYRRCRVPGVGVPGVGGATGSDVNFDLSTATAKTYTKFIEDFRATLPFSHKVYDIPLLYSTISDSRRFILLNLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVNVPGVGVPGVGHGVSGHGQHGVHG

SEQ ID NO: 35 - protein, 336 aa, Artificial Sequence
  An example of fusion proteins according to the present invention
VPGVGVPGVGFLPLLAGLAANFLPTIICFISYKCVPGVGVPGVGGATGSDVNFDLSTATAKTYTKFIEDFRATLPFSHKVYDIPLLYSTISDSRRFILLNLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVNVPGVGVPGVGKLAKLAKKLAKLAK

SEQ ID NO: 36 - protein, 325 aa, Artificial Sequence
  Polypeptide sequence of RetroGAD1
GICRCIGRGICRCICGRVPGVGVPGVGGATGSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKGLWSKIKEAAKAAGKAALNAVTGLVNQGDQPSVPGVGVPGVG

SEQ ID NO: 37 - DNA, 1185 bp, Artificial Sequence
  Coding sequence of Amatilin
gggcagtgagcggaaggcccatgaggccagttaattaagaggtaccgaattctcattcggtttgtgtagattgagaagaggtttctgtgctcacggtagatgtagattcccatccatcccaatcggtagatgttccagattcgttcagtgttgtagaagagtttgggtcccaggtgttggtgttccaggtgttggaggtgctactggttctgatgttaacttcgacttgtccactgctactgctaagacttacactaagttcatcgaggacttcagagctactttgccattctcccacaaggtttacgacatccctttgttgtactccactatctccgactccagaagattcatcttgttgaacttgacttcctacgcttacgagactatctccgttgctatcgacgttacaaacgtttacgttgttgcttacagaactagagatgtttcctacttcttcaaagagtccccaccagaggcttacaacatcttgttcaagggtactagaaagatcactttgccatacactggtaactacgagaacttgcagactgctgctcacaagatcagagagaacatcgacttgggtttgccagctttgtcctccgctatcactactttgttctactacaacgctcagtccgctccatccgctttgttggttttgatccagactactgctgaggctgctagattcaagtacatcgagagacacgttgctaagtacgttgctacaaacttcaagccaaacttggctatcatctccttggagaaccagtggtctgctttgtccaagcagatcttcttggctcaaaaccagggtggtaagttcagaaacccagtcgacttgatcaagccaaccggtgagagattccaggttactaatgttgactccgacgttgttaagggtaacatcaagttgttgttgaactccagagcttccactgctgacgagaacttcatcactactatgactttgttgggtgagtccgttgttaactcctgtgcttccagatgtaagggtcactgtagagctagaagatgtggttactacgtttccgttctgtacagaggtagatgttactgtaaatgtttgagatgtgtccccggtgttggagtccctggtgtcggtgcggccgcgagctcatggcgcgcctaggccttgacggccttccgccaattcgc

SEQ ID NO: 38 - DNA, 1084 bp, Artificial Sequence
  Coding sequence for RetroGAD1
cgaattggcggaaggccgtcaaggccacgtgtcttgtccaggtaccgaattcggaatctgtagatgcatctgcggtagaggtatctgcagatgtatttgtggaagagtcccaggtgttggtgttccaggtgttggaggtgctactggttctggtttggacactgtttcattctccactaagggtgctacttacatcacttacgttaactttttgaacgagttgagagttaagttgaagccagagggtaactcccacggtatccctttgttgagaaagaagtgtgacgacccaggtaagtgtttcgttttggttgctttgtccaacgacaacggtcagttggctgagattgctatcgacgttacttccgtttacgttgttggttaccaggttagaaacagatcctacttcttcaaggacgctccagacgctgcttacgaaggtttgttcaagaacactatcaagactagattgcacttcggtggttcctacccatctttggaaggtgagaaggcttacagagagactactgacttgggtatcgagccattgagaatcggtatcaagaagttggacgagaacgctatcgacaactacaagccaactgagatcgcttcctccttgttggttgttatccagatggtttccgaggctgctagattcactttcatcgagaaccagatcagaaacaacttccagcagagaatcagaccagctaacaacactatttccttggagaacaagtggggtaagttgtccttccagatcagaacatccggtgctaacggtatgttctctgaggctgttgagttggagagagctaacggtaagaagtactacgttactgctgttgaccaggttaagccaaagatcgctttgttgaagttcgttgacaaggacccaaagggtttgtggtccaagatcaaagaggctgctaaggctgctggtaaggctgctttgaatgctgttactggtttggttaaccagggtgaccaaccatctgtccctggtgttggagtccctggtgtcggtgcggccgcgagctctggagcacaagactggcctcatgggccttccgctcactgc

SEQ ID NO: 39 - DNA, 998 bp, Artificial Sequence
  Coding sequence of Tampal 1
ggatccgttccgggtgtgggtgttccgggtgttggtaaatggtgtttcgtgtttgttatcgcggtatttgttatcgtcgttgtcgtgtgccaggcgttggcgttccaggcgtgggtggtgcaaccggtagtgatgttaattttgatctgagcaccgcaaccgcaaaaacctataccaaatttatcgaagattttcgtgcaaccctgccgtttagccataaagtttatgatattccgctgctgtatagcaccattagcgatagccgtcgttttattctgctgaatctgaccagctatgcctatgaaaccattagcgttgcaattgatgtgaccaatgtttatgttgttgcatatcgtacccgtgatgtgagctattttttcaaagaaagccctccggaagcctataacattctgtttaaaggcacccgcaaaatcaccctgccgtataccggtaattatgaaaatctgcagaccgcagcacataaaattcgcgaaaatattgatctgggtctgcctgcactgagcagcgcaattaccaccctgttttattacaatgcacagagcgcaccgagcgcactgctggttctgattcagaccaccgcagaagcagcacgctttaaatacattgaacgtcatgttgccaaatacgtggccaccaactttaaaccgaatctggcaattattagcctggaaaatcagtggtcagcactgagcaaacaaatttttctggcacagaatcagggtggcaaatttcgtaatccggttgatctgattaaaccgaccggtgaacgttttcaggttaccaatgttgatagtgatgtggtgaaaggcaacattaaactgctgctgaatagccgtgcaagcaccgcagatgaaaactttattaccaccatgaccctgctgggtgaaagcgttgttaatgttcctggtgttggcgtgcctggtgttggtcatggtgttagcggtcatggtcagcatggtgttcatggttaaaagctt

SEQ ID NO: 40 - DNA, 20 bp, Artificial Sequence
  Sense Primer F1 of Example 12
atggatttggcaacctaaca

SEQ ID NO: 41 - DNA, 19 bp, Artificial Sequence
  Sense primer F2 of Example 12
aattcgtggagagaggtcc

SEQ ID NO: 42 - DNA, 20 bp, Artificial Sequence
  Sense primer F3 of Example 12
atctctaccgtcacacagcc

SEQ ID NO: 43 - DNA, 23 bp, Artificial Sequence
  Antisense primer R1 of Example 12
gaagattttaatgtccttgctcg